(19) United States
(12) Patent Application Publication
(10) Pub. No.: US 2005/0032777 A1
(43) Pub. Date: Feb. 10, 2005

(54) Diazacycloalkanes as oxytocin agonists

(76) Inventors: Peter Jeremy Hudson, Copenhagen (DK); Andrezej Roman Batt, Southampton (GB); Gary Robert William Pitt, Hampshire (GB); David Philip Rooker, Hampshire (GB); Celine Marguerite Simone Heeney, Southampton (GB); Michael Bryan Roe, Southampton (GB)

Correspondence Address: FOLEY AND LARDNER;SUITE 500, 3000 K STREET NW, WASHINGTON, DC, 20007, US

(21) Appl. No.: 10/486,715

(22) PCT Filed: Aug. 6, 2002
(86) PCT No.: PCT/GB02/03593

(30) Foreign Application Priority Data
Aug. 16, 2001 (GB) 0120051.8

(51) Int. Cl. A61K 31/551; A61K 31/55; A61K 31/519

(52) U.S. Cl. 514/215; 514/221; 514/220; 514/252.16; 514/261.1; 540/558; 540/578

(57) ABSTRACT

Compounds according to general formula (1), wherein G¹ is NR⁵R⁶ or a fused polycyclic group are novel. They are selective and potent oxytocin agonists. Pharmaceutical compositions of such compounds are useful in the treatment of, inter alia, erectile dysfunction.

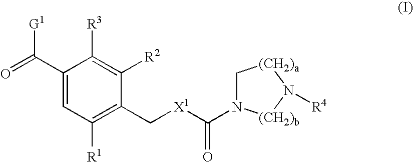

### FIELD OF THE INVENTION

The present invention relates to a series of non-peptide oxytocin agonists and to pharmaceutical compositions comprising such compounds. The compositions are useful for the treatment of certain physiological disorders, such as erectile dysfunction.

### BACKGROUND

Neurophyseal Hormones

The neurophyseal hormones oxytocin (OT) and vasopressin (VP) are cyclic nonapeptides secreted by the posterior pituitary gland. The structure of oxytocin is shown below.

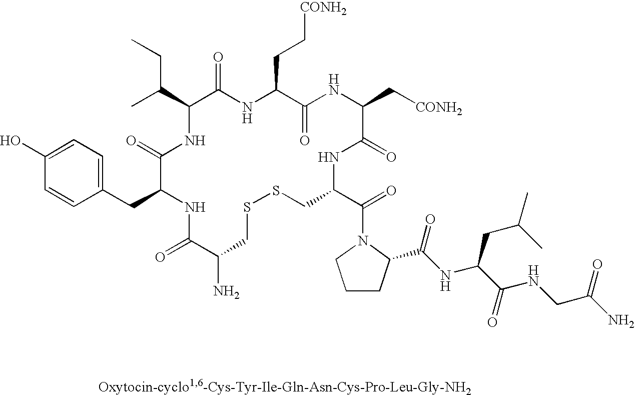

Vasopressin differs from oxytocin in that it has phenylalanine at position 3 in place of isoleucine and arginine at position 8 in place of leucine. Both hormones are synthesised in vivo as larger precursors, neurophysins, which are subject to post-translational processing to release the mature peptides. OT and VP act through a family of heptahelical receptors.

The first target organs to be identified for OT were the uterus, where it is implicated in the onset and progress of labour, and mammary glands, where it is involved in the regulation of milk expression. Other organs also express OT receptors, and it is clear that OT has a range of physiological roles that have not been fully elaborated yet. In particular, it has been suggested that OT acting in the CNS is involved in the erectile response in males, and in the regulation of female sexual arousal. For example, OT is erectogenic when administered i.c.v. to male rats. It also has erectogenic activity when given iv., but the doses required are up to two orders of magnitude greater, which is consistent with a central mode of action.

Oxytocin Agonists and Antagonists

A number of peptide analogues of OT are known in the literature. These include both agonists and antagonists. OT and its agonists are used, for example, to accelerate labour and to increase uterine muscle tone to control post-partum bleeding, and one antagonist, atosiban, has recently been registered as a treatment for pre-term labour. However, the peptidic nature of these compounds means that they are not likely to be bioavailable after oral dosing or to cross efficiently into the CNS. In order to get drugs that can be given orally and to be able to exploit the central effects of OT, attention has increasingly turned to non-peptides. As a result, there are many publications describing non-peptide OT antagonists in early-stage development. So far, however, there have been no reports of non-peptide OT agonists. This is not unexpected, as it is generally held that it is easier to find a receptor antagonist than an agonist.

So there remains a need for non-peptide OT receptor agonists. Such compounds should preferably be selective for the OT receptor over the VP receptors. They could be expected to show therapeutic utility in male and female sexual dysfunction, particularly male erectile dysfunction, in promoting labour, in controlling post-partum bleeding, in increasing milk let-down as well as a number of other indications.

### SUMMARY OF THE INVENTION

We describe herein a series of potent and specific OT receptor agonists. In a first aspect, the present invention comprises novel compounds according to general formula 1, and pharmaceutically acceptable salts thereof. 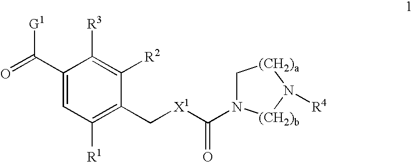
G¹ is a group according to general formula 2, 3, 4, 5, 6 or 7. 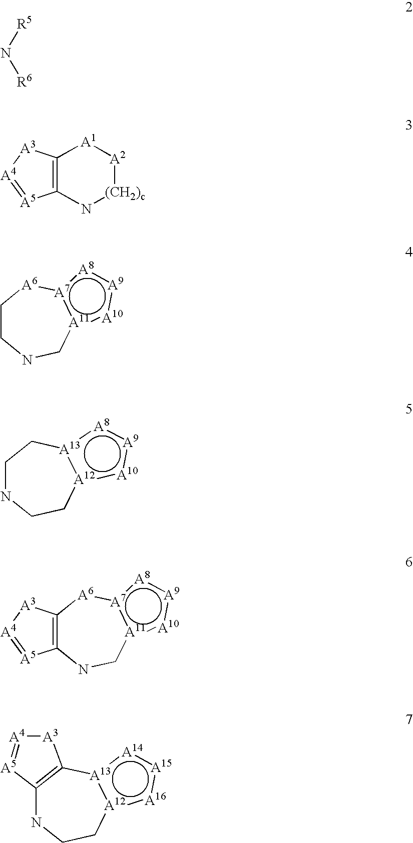
A¹ is CH₂, CH(OH), NH, N-alkyl, O or S; A² is CH₂, CH(OH), C(═O) or NH; A ³ is S, NH, N-alkyl, —CH═CH— or —CH═N—; A⁴ and A⁵ are each CH or N; A⁶ is CH₂, NH, N-alkyl or O; A⁷ and A¹¹ are C or N; A⁸ and A⁹ are CH, N, NH, N(CH₂)_{d}R⁷ or S; A¹⁰ is —OH═COH—, CH, N, NH, N—(CH₂)_{d}—R⁷ or S; A¹² and A¹³ are N or C and A¹⁴, A¹⁵ and A¹⁶ are NH, N—CH₃, S, N or CH, provided that not more than one of A⁸, A⁹ and A¹⁰ is NH, N—(CH₂)_{d}—R⁷ or S; that A⁷ and A¹¹ are not both simultaneously N; that neither A⁷ nor A¹¹ is N if one of A⁸, A⁹ and A¹⁰ is NH, N—(CH₂)_{d}—R⁷ or S; that if A¹⁰ is —CH═CH— then A⁸ is N, A⁹ is CH and both A⁷ and A¹¹ are C; that if A¹⁰ is not —CH═CH— then one of A⁸, A⁹ and A¹⁰ is NH, N—(CH₂)_{d}—R⁷ or S or one of A⁷ and A¹¹ is N; that not more than one of A¹⁴, A¹⁵ and A¹⁶ is NH, N—CH₃ or S; that A¹² and A¹³ are not both simultaneously N; that if one of A¹⁴, A¹⁵ and A¹⁶ is NH, N—CH₃ or S then A¹² and A¹³ are both C; and that one of A¹⁴, A¹⁵ and A¹⁶ is NH, N—CH₃ or S or one of A¹² and A¹³ is N.
X¹ is O or NH.
R¹, R² and R³ are each H, alkyl, O-alkyl, F, Cl or Br.
R⁴ is H, alkyl, optionally substituted phenyl, pyridyl, thienyl or furyl, or is (CH₂)ₑ—R⁸.
R⁵ and R⁶ are each independently alkyl, Ar or CH₂)_{f}—Ar, where Ar is optionally substituted phenyl or thienyl.
R⁷ and R⁸ are each independently H, alkyl, optionally substituted phenyl, pyridyl, thienyl or furyl, F, OH, O-alkyl, S-alkyl, O-acyl, NH₂, NH-alkyl, N(alkyl)₂, NH-acyl, N(alkyl)-acyl, CO₂H, CO₂-alkyl, CONH₂, CONH-alkyl, CON(alkyl)₂, CN or CF₃.
a is 1 or 2, b is 1, 2 or 3, c is 1 or 2, d is 1, 2 or 3; e is 1, 2 or 3 and f is 1, 2 or 3.

In a second aspect, the present invention comprises pharmaceutical compositions of these novel compounds, which compositions are useful for the treatment of, inter alia, male erectile dysfunction. In further aspects, the present invention comprises the use of such compositions in therapy and therapeutic methods using the compositions.

### DETAILED DESCRIPTION OF THE INVENTION

In a first aspect, the present invention comprises novel benzyl carbamates and ureas according to general formula 1.

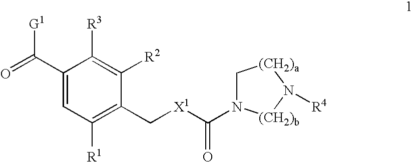

In this general formula the substituents R¹, R² and R³ are independently selected from hydrogen (H), alkyl groups, alkoxy (O-alkyl) groups, and the halogens fluorine (F), chlorine (Cl) and bromine (Br). Preferably, at least one of R¹, R² and R³ is H and at least one is not H. More preferably, one of R¹, R² and R³ is an alkyl group or a halogen and the others are H. Most preferably, R¹ is methyl or C¹ and R² and R³ are both H.

The linking group X¹ is selected from oxygen (O) and unsubstituted nitrogen (NH). Preferably, X¹ is NH.

The integer a may be 1 or 2, and the integer b may be 1, 2 or 3. Preferably a is 1 and b is 2 such that this ring is a piperazine.

The substituent R⁴ is selected from H, alkyl groups, optionally substituted phenyl, pyridyl, thienyl, furyl, pyrrolyl, pyrazolyl, imidazolyl, oxazolyl, isoxazolyl, thiazolyl and isothiazolyl groups, a group-(CO)—O—(CH₂)ₑR⁸ where e is 1, 2, 3 or 4, a group —CH₂)ₑR⁸, where e is 1, 2, 3 or 4, —CH₂—CH═CH—CH₂—R⁸, —CH₂—C≡C—CH₂—R⁸, —(CH₂)_{g}—CH(OH)—(CH₂)ₕ—R⁸, where g and h are independently 1 or 2,
—(CH₂)ᵢ—O—(CH₂)ⱼ—R⁸ where i and j are independently 1 or 2, and 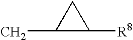
R²⁸ is selected from H, F, CF₃, alkyl groups, O-alkyl groups, S-alkyl groups, O-acyl groups, hydroxyalkyl groups, amino groups such as NH₂, NH-alkyl, N(alkyl)₂, 1-pyrrolidinyl, 1-piperidinyl and 4-morpholinyl, NH-acyl, N(alkyl)-acyl, CO₂H, CO₂-alkyl, CONH₂, CONH-alkyl, CON(alkyl)₂, CN and optionally substituted phenyl, pyridyl, thienyl, furyl, pyrrolyl, pyrazolyl, imidazolyl, oxazolyl, isoxazolyl, thiazolyl and isothiazolyl groups. Suitable optional substituents for the phenyl, pyridyl, thienyl, furyl, pyrrolyl, pyrazolyl, imidazolyl, oxazolyl, isoxazolyl, thiazolyl and isothiazolyl groups in R⁴ and R⁸ include F, Cl, Br, CF₃, alkyl groups, OH, O-alkyl groups, hydroxyalkyl groups, amino groups such as NH₂, NH-alkyl and N(alkyl)₂, NH-acyl, N(alkyl)-acyl, CO₂H, CO₂-alkyl, CONH₂, CONH-alkyl, CON(alkyl)₂, oxadiazolyl, thiadiazolyl, CN and NO₂. The phenyl, pyridyl, thienyl furyl, pyrrolyl, pyrazolyl, imidazolyl, oxazolyl, isoxazolyl, thiazolyl or isothiazolyl group may have up to three such substituents which may be the same or different.

The group G¹ is a disubstituted nitrogen such that the C(═O)-G¹ bond is an amide bond. G¹ is selected from an acyclic group according to general formula 2, a fused bicyclic group according to general formulae 3, 4 and 5, and a fused tricyclic group according to general formulae 6 and 7,

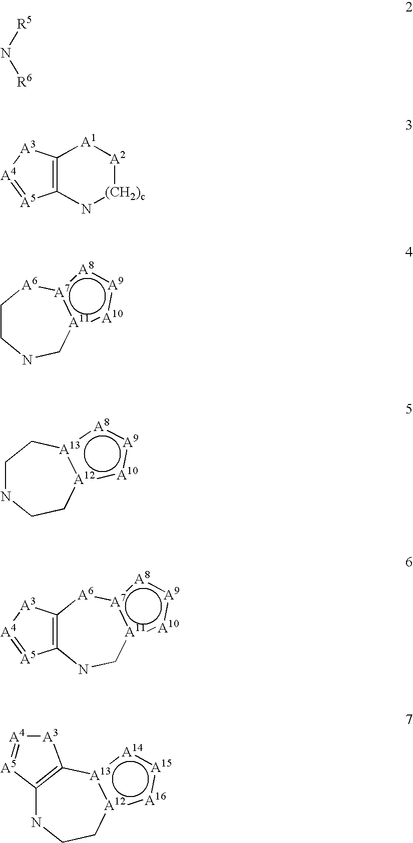

In general formula 2, R⁵ and R⁶ are independently selected from alkyl, Ar and —(CH₂)_{f}—Ar, where f is 1, 2 or 3 and Ar is selected from thienyl and optionally substituted phenyl. Suitable substituents for the phenyl group are alkyl groups, OH, alkoxy groups, halogens, NH₂, NH-alkyl and N(alkyl)₂. The phenyl group may be substituted with up to three such substituents which may be the same or different.

In general formula 3, A¹ is selected from CH₂, CH(OH), NH, N-alkyl, O and S. A² is selected from CH₂, CH(OH), C(═O) and NH, and c is 1 or 2, preferably 2. It is preferred that when A² is NH then A¹ is CH₂. It is also preferred that when A² is C(═O) then A¹ is NH or N-alkyl.

In general formulae 3, 6 and 7, A³ is selected from S, NH, N-alkyl, —CH═CH— and —CH═N— and A⁴ and A⁵ are each selected from CH and N. In a preferred embodiment, A³ is S and A⁴ and A⁵ are both CH, so as to form a thiophene ring. In another preferred embodiment, A³ is —CH═CH— and A⁴ and As are both CH, so as to form a benzene ring. In another preferred embodiment, A³ is —CH═N— and A⁴ and As are both CH, so as to form a pyridine ring. In another preferred embodiment, A³ is —CH═CH—, A⁴ is CH and A⁵ is N, again so as to form a pyridine ring.

In general formulae 4 and 6, Ar is selected from CH₂, NH, N-alkyl and 0, A⁷ and A¹¹ are selected from C and N, A⁸ and A⁹ are selected from CH, N, NH, N—(CH₂)_{d}—R⁷ and S and A¹⁰ is selected from —CH═CH—, CH, N, NH, N—(CH₂)_{d}—R⁷ and S, where d is 1, 2 or 3 and R⁷ is selected from H, F, CF₃, alkyl groups, OH, O-alkyl groups, S-alkyl groups, O-acyl groups, amino groups such as NH₂, NH-alkyl and N(alkyl)₂, NH-acyl, N(alkyl)-acyl, CO₂H, CO₂-alkyl, CONH₂, CONH-alkyl, CON(alkyl)₂, CN and optionally substituted phenyl groups. Suitable optional substituents for the phenyl groups in R⁷ include F, Cl, Br, CF₃, alkyl groups, O-alkyl groups, amino groups such as NH₂, NH-alkyl and N(alkyl)₂, NH-acyl, N(alkyl)-acyl, CO₂H, CO₂-alkyl, CONH₂, CONH-alkyl, CON(alkyl)₂, CN and NO₂. The phenyl group may have up to three such substituents which may be the same or different.

The ring constituted by A⁷, A⁸, A⁹, A¹⁰ and A¹¹ is aromatic, and accordingly the groups must satisfy certain requirements. When A¹⁰ is —CH═CH— the ring is a six-membered ring. As such, it can only comprise atoms of the type —C(R)=and —N═. Hence A⁷ and A¹¹ must both be C and A⁸ and A⁹ must be either CH or N. We have found that suitable activity is only obtained when A⁸ is N and A⁹ is CH. When A¹⁰ is not —CH═CH— then the ring is a five-membered ring. In this case one, and only one, of the atoms in the ring must be S or a trigonal nitrogen. In this context, a “trigonal nitrogen” is a nitrogen atom linked covalently to three different atoms. Two of these atoms are the immediate neighbours to the nitrogen atom in the five-membered ring. The third is a hydrogen, carbon or other atom linked to the five-membered ring. Thus it follows that, when A¹⁰ is not —CH═CH— then one (and only one) of A⁷, A⁸, A⁹, A¹⁰ and A¹¹ must be S or a trigonal nitrogen. Hence the selection of A⁷, A⁸, A⁹, A¹⁰ and A¹¹ is subject to the following restrictions.
1) If A¹⁰ is not —CH═CH— then one of A⁸, A⁹ and A¹⁰ is NH, N—(CH₂)_{d}—R⁷ or S or one of A⁷ and A¹ is N.
2) Not more than one of A⁸, A⁹ and A¹⁰ may be NH, N—(CH₂)_{d}—R⁷ or S.
3) A⁷ and A¹¹ may not both simultaneously be N.
4) Neither A⁷ nor All may be N if one of A⁸, A⁹ and A¹⁰ is NH, N(CH₂)_{d}—R⁷ or S.
   In a preferred embodiment, A⁶ is NH. In another preferred embodiment, A⁸ is NH or N—(CH₂)_{d}—R⁷. In a more preferred embodiment, A⁸ is NH or N—(CH₂)_{d}—R⁷, A⁹ is N and A¹⁰ is CH.

In general formulae 5 and 7, A¹² and A¹³ are selected from N and C and A¹⁴, A¹⁵ and A¹⁶ are selected from NH, N—CH₃, S, N and CH. Again, these atoms constitute an aromatic five-membered ring and so there must be one, and only one, S or trigonal nitrogen. Hence the selection of A¹², A¹³, A¹⁴, A¹⁵ and A¹⁶ is subject to the following restrictions.
1) One of A¹⁴, A¹⁵ and A¹⁶ is NH, N—CH₃ or S or one of A¹² and A¹³ is N.
2) Not more than one of A¹⁴, A¹⁵ and A¹⁶ is NH, N—CH₃ or S.
3) A¹² and A¹³ may not both simultaneously be N.
4) If one of A¹⁴, A¹⁵ and A¹⁶ is NH, N—CH₃ or S then A¹² and A¹³ are both C
   As used herein, the term “alkyl” is intended to designate lower alkyl groups, i.e. saturated hydrocarbon groups of between one and six carbon atoms, including linear, branched and cyclic alkyl groups. Examples of “alkyl” include, but are not limited to: C₁-methyl, C₂-ethyl, C₃-propyl, isopropyl, cyclopropyl, C₄-n-butyl, sec-butyl, isobutyl, tert-butyl, cyclobutyl, cyclopropylmethyl, methylcyclopropyl, C₅-n-pentyl, neopentyl, cyclopropylethyl, dimethylcyclopropyl, and C₆-n-hexyl, cyclohexyl, bicyclo[3.1.0]hexyl.

The term “alkenyl” denotes a lower alkenyl group, i.e. a mono-unsaturated hydrocarbon group of between two and six carbon atoms, including linear, branched and cyclic alkenyl groups. Examples of “alkenyl” include, but are not limited to: C₂-vinyl, C₃-allyl, 1-methylvinyl, 1-propenyl, C₄-but-3-enyl, but-2-enyl, methallyl.

The term “alkynyl” denotes a lower alkynyl group, i.e. an unsaturated hydrocarbon group of between two and six carbon atoms which includes a carbon-carbon triple bond, including linear, branched and cyclic alkynyl groups. Examples of “alkynyl” include, but are not limited to: C₂-ethynyl, C₃-propargyl, 1-propynyl.

The term “hydroxyalkyl” denotes an alkyl group as defined above in which one or more of the hydrogen atoms are replaced by hydroxyl groups (OH). In general, not more than one hydroxyl group will be attached to any particular carbon atom within the hydroxalkyl group. Examples of hydroxyalkyl groups include, but are not limited to: hydroxymethyl (HOCH₂), 1-hydroxyethyl (CH₃CH(OH)), 2-hydroxyethyl (HOCH₂CH₂), 1,2-dihydroxyethyl (HOCH₂CH(OH)) 4-hydroxy-2-pentyl (CH₃CH(OH)CH₂CH(CH₃)), and 4-hydroxy-cyclohexyl.

The term “acyl” denotes a group R—C(═O), where R is H, a saturated or unsaturated hydrocarbon moiety of up to seven carbon atoms or a pyridyl or thienyl group. Examples of acyl groups include, but are not limited to: formyl, acetyl, pivaloyl, benzoyl and nicotinoyl.

The compounds according to the present invention generally contain a basic nitrogen atom and so are capable of forming addition salts with protic acids such as hydrochloric acid, sulphuric acid, phosphoric acid, acetic acid, trifluoroacetic acid, benzoic acid, maleic acid, citric acid, fumaric acid, methanesulphonic acid and the like. The compounds of the present invention may also contain an acidic group, such as a carboxylic acid group at R⁷ or R⁸ These compounds may exist as inner salts (zwitterions) or as salts such as sodium, potassium, magnesium, calcium or tetra-alkylammonium salts. To the extent that such salts are pharmaceutically acceptable, they are included within the scope of the present invention.

The compounds according to the present invention may have one or more stereogenic centres (“asymmetric carbon atoms”) and so may exhibit optical isomerism. The scope of the present invention includes all epimers, enantiomers and diastereomers of compounds according to general formula 1, including single isomers, mixtures and racemates.
Particularly preferred embodiments within the present invention are those compounds that combine two or more of the preferred features described above. One such particularly preferred embodiment is a urea according to general formula 8.

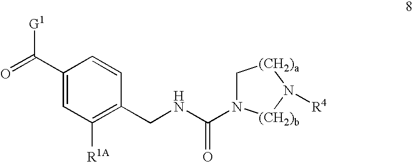

In general formula 8, R^{1A} is methyl or Cl. G¹, R⁴, a and b are as previously defined.
More preferred is a urea according to general formula 9.

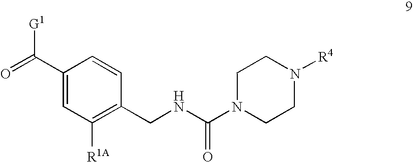

In general formula 9, R^{1A}, R⁴ and G¹ are as previously defined.
Another particularly preferred embodiment is a compound according to general formula 10, which corresponds to a compound according to general formula I in which G¹ is a group according to general formula 6 wherein A⁴, A⁵ and A¹⁰ are all CH, A⁶ is NH, A⁷ and A¹¹ are both C, A⁸ is N(CH₂)_{d}R⁷ and A⁹ is N.

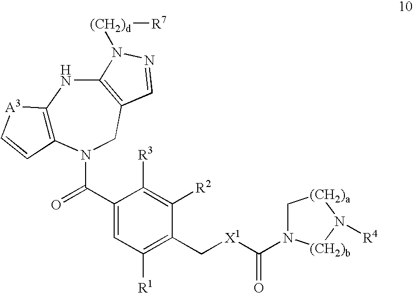

In general formula 10, R¹, R², R³, R⁴, R⁵, A³, X¹, a, b and d are as previously defined.

A most preferred embodiment is a compound according to general formula 11.

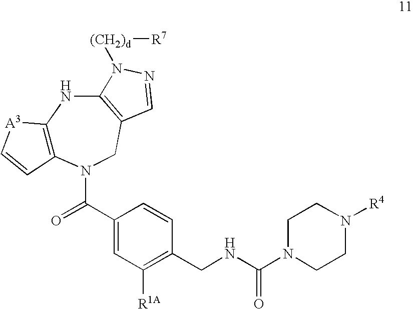

In general formula 11, R^{1A}, R⁴, R⁷, A³ and d are as previously defined.

Individual preferred compounds within the invention include:
5-(4-(4-cyclopropylmethylpiperazine-1-carbonylaminomethyl)-3-methylbenzoyl)-1-methyl-4,10-dihydropyrazolo[5,4-b][1,5]benzodiazepine,
5-(4-(4-benzylpiperazine-1-carbonylaminomethyl)-3-methylbenzoyl)-1-methyl-4,10-dihydropyrazolo[5,4-b][1,5]benzodiazepine,
5-(4-(4-(3-hydroxybenzyl)piperazine-1-carbonylaminomethyl)-3-methylbenzoyl)-1-methyl-4,10-dihydropyrazolo[5,4-b][1 ₁5]benzodiazepine,
5-(4-(4-(3-hydroxymethylbenzyl)piperazine-1-carbonylaminomethyl)-3-methylbenzoyl)-1-methyl-4,10-dihydropyrazolo[5,4-b][1,5]benzodiazepine.
1-methyl-5-(3-methyl-4-(4-(4-picolyl)piperazine-1-carbonylaminomethyl)benzoyl)_{4,10}-dihydropyrazolo[5,4-b][1,5]benzodiazepine,
5-(4-(4-(2-hydroxyethyl)piperazine-1-carbonylaminomethyl)-3-methylbenzoyl)-1-methyl-4,10-dihydropyrazolo[5,4-b][1,5]benzodiazepine,
1-methyl-5-(3-methyl-4-(4-(3-(methylthio)propyl)piperazine-1-carbonylaminomethyl)benzoyl)-4,10-dihydropyrazolo[5,4-b][1,5]benzodiazepine,
5-(4-(4-(2-aminoethyl)piperazine-1-carbonylaminomethyl)-3-methylbenzoyl)-1-methyl-4,10-dihydropyrazolo[5,4-b][1,5]benzodiazepine, and
5-(4-(4-(2-hydroxyethyl)piperazine-1-carbonylaminomethyl)-3-methylbenzoyl)-1-methyl-4,10-dihydropyrazolo[4,5-c]pyrido[2,3-b][1,4]diazepine.

The compounds of the present invention can be prepared by standard chemical manipulations. In general, compounds according to general formula 1 can be considered to consist of three component parts:
Component C¹ corresponding to G¹
Component C² corresponding to the substituted benzoyl unit
Component C³ corresponding to the saturated heterocycle

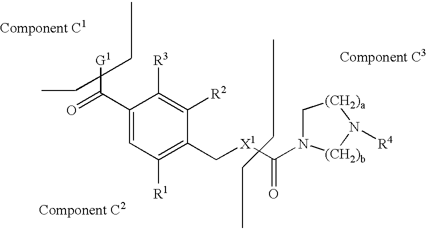

Intermediates corresponding to these components are prepared and then assembled to give the final product. These three components are:

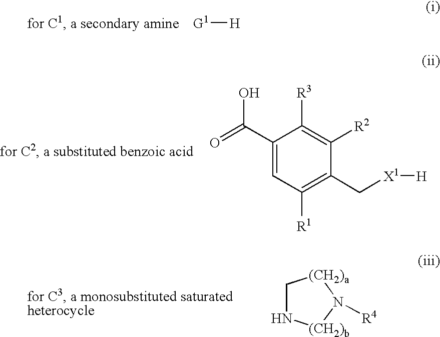

It will be recognised that the substituted benzoic acid that serves for C² has two functional groups, one of which will need temporary protection during the assembly of the final compound. The principles of functional group protection are well known in the art and are described in, for example, J. F. W. McOmie, “Protective Groups in Organic Chemistry”, Plenum Press, 1973; T. W. Greene and P. G. M. Wuts, “Protective Groups in Organic Synthesis”, 2^{nd} edition, John Wiley, 1991; and P. J. Kocienski, “Protecting groups”, Georg Thieme Verlag, 1994. The carboxylic acid group will usually be protected as an ester, such as the methyl, benzyl or tert-butyl ester. The primary amine of the benzoic acid (when X¹═NH) will usually be protected as a carbamate derivative such as the tert-butyl carbamate (BOC derivative), the benzyl carbamate (CBZ or more simply Z derivative) or the 9-fluorenylmethyl carbamate (Fmoc derivative). When X¹═O the resulting alcohol function will usually be protected as an ester such as an acetate, or an ether such as a methoxymethyl, tetrahydropyranyl or trialkylsilyl ether. Other functional groups may require protection. For example, the group G¹ may include one or more primary or secondary amino groups which may need protection. In the following general description of the synthetic methodology it will be assumed that such protection is used when necessary.

(i) Preparation of Secondary Amine for C¹

Acyclic secondary amines corresponding to HNR⁵R⁵ are well known. Many are items of commerce. Those that are not may be prepared according to published methods or by simple modification of such methods. Some particularly useful methods are listed below.

a) Alkylation 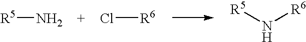
(This method is only applicable in cases where further alkylation can be avoided.)

b) Reductive Amination 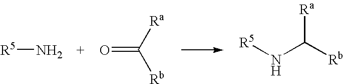
(where R^{a}CHR^{b} corresponds to R⁶)

c) Amide Reduction 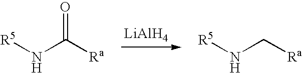
(where R^{a}CH₂ corresponds to R⁶)

The starting amide can itself be prepared using well known methods.

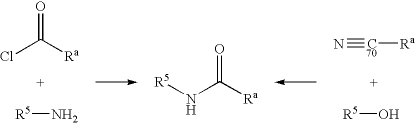

Secondary amines corresponding to C¹ where G¹ is a group according to general formulae 3-7 are generally not commercially available. They can be prepared according to published methods, or by obvious modifications of such methods. Particularly useful methods are described in: Aranapakam et al., Bioorg. Med. Chem. Lett. 1993, 1733; Artico et al., Farmaco. Ed. Sci. 24, 1969, 276; Artico et al., Farmaco. Ed. Sci. 32, 1977, 339; Chakrabarti et al., J. Med. Chem. 23, 1980, 878; Chakrabarti et al., J. Med. Chem. 23, 1980, 884; Chakrabarti et al., J. Med. Chem. 32, 1989, 2573; Chimirri et al., Heterocycles 36, 1993, 601; Grunewald et al., J. Med. Chem. 39, 1996, 3539; Klunder et al., J. Med. Chem. 35, 1992, 1887; Liegeois et al., J. Med. Chem. 37, 1994, 519; Olagbemiro et al., J. Het. Chem. 19, 1982, 1501; Wright et al., J. Med. Chem. 23, 1980, 462; Yamamoto et al., Tet. Lett. 24, 1983, 4711; and International patent application, publication number WO99/06403.

(ii) Preparation of Substituted Benzoic Acid for C²

Substituted benzoic acids corresponding to C² are not generally items of commerce, but they can be prepared using published methods or obvious variations of such methods. The main challenge is generally the elaboration of the —CH₂X¹H functionality at the 4-position. Some useful transformations are listed below.

a) Bromination/Substitution 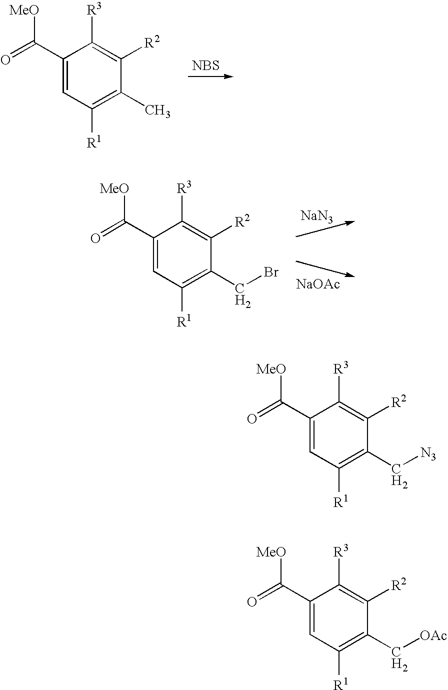
b) Sandmeyer Reaction/Reduction 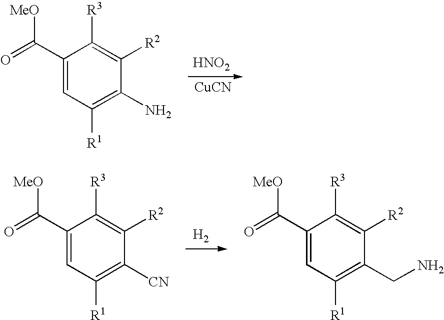
(iii) Preparation of Heterocycle Derivative for C³

Certain heterocycles corresponding to C³, particularly N-aryl piperazines, are items of commerce. Other heterocycles can be prepared according to the methods described in the literature. Useful transformations include the following.

a) Alkylation or Reductive Alkylation 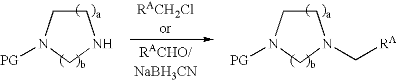
(where PG is a protecting group and R^{A}CH₂ is R⁴)

b) Acylation/Reduction

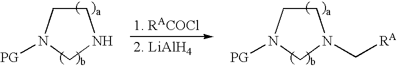

c) Reduction

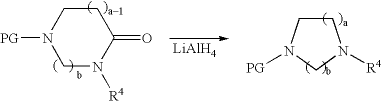

With the three components, suitably protected if necessary, in hand, the assembly of the final compound requires the formation of two bonds: between C¹ and C², and between C² and C³. These bond-forming steps may be taken in either order. Thus, the following sequences can be proposed:
C¹+C²→C¹C²→C¹C²C³
C²+C³→C²C³→C¹C²C³
(i) Formation of C¹-C² Bond

The bond between C¹ and C² is a simple amide bond. The chemistry for making such bonds from a carboxylic acid and a secondary amine is well known in the art of organic synthesis, and particularly in the field of peptide synthesis. The carboxylic acid may be converted into a more reactive species such as an acid chloride (using, for example oxalyl chloride or thionyl chloride) or a mixed anhydride (using isobutyl chloroformate). This reactive species is then added to the secondary amine in a suitable solvent, generally an aprotic solvent such as dichloromethane or dimethylformamide, in the presence of a base such as triethylamine or 4-dimethylaminopyridine, and the reaction is allowed to proceed at a temperature between −20° C. and the boiling point of the solvent. The choice of temperature and the time allowed for the reaction will depend on the reactivity of the two components.

Alternatively, the carboxylic acid and the secondary amine may be mixed in a suitable solvent as above, optionally in the presence of a base, and a condensing agent added. Suitable condensing agents include carbodiimides, such as dicyclohexylcarbodiimide (DCC) and N-ethyl-N′-dimethylaminopropylcarbodiimide (EDC, also WSCD for water-soluble carbodiimide), phosphorus reagents such as (benzotriazol-1-yloxy)-tris(dimethylamino)phosphonium hexafluorophosphate (BOP), (benzotriazol-1-yloxy)-tripyrrolidinophosphonium hexafluorophosphate (PyBOP®) and bromotripyrrolidino-phosphonium hexafluorophosphate (PyBroP®), and ureas such as O-(benzotriazol-1-yl)-N,NN′, NN′-tetramethyluronium hexafluorophosphate (HBTU).

(ii) Formation of C₂-C₃ Bond

The bond between C² and C³ is a carbamate (when X¹═O) or a urea (when X¹═NH). The first step in the formation of this bond is generally to react the heterocycle derivative with phosgene or a phosgene equivalent such as trichloromethyl chloroformate, bis(trichloromethyl)carbonate or carbonyldiimidazole. Again, an aprotic solvent and a tertiary amine base will generally be used. The intermediate formed in this step is usually not isolated. The alcohol (X¹═O) or amine (X¹═NH) is added and the reaction is allowed to continue, directly forming the carbamate or urea. As an alternative, when X¹═NH the reactive intermediate may be formed by the reaction of c2 with the phosgene equivalent and the amine added in the second part of the synthesis.

The compounds according to the present invention are useful in human and animal therapy. When so used, they will generally be formulated in an appropriate manner. Thus a second aspect of the present invention is a pharmaceutical formulation that includes a compound as described above as an active ingredient. A third aspect of the present invention is the use of a compound according to the first aspect in the manufacture of such a composition.

The composition according to the present invention may be presented in any form that is known in the art. For example, the formulation may be presented as a tablet, capsule, powder, suppository, cream, solution or suspension, or in a more complex form such as an adhesive patch. The formulation will generally include one or more excipients, such as diluents, bulking agents, binding agents, dispersants, solvents, preservatives, flavoring agents and the like. Where the formulation is presented as a tablet or capsule the excipients may optionally include one or more agents to control the release of the active species, such as a coating of a polymer that is insoluble at low pH but soluble at neutral or high pH. Such a coating (known as an “enteric coating”) prevents the release of the active agent in the stomach but allows its release in the intestines. The formulation may also include one or more additional pharmacologically active species. Preferably the formulation includes no such additional active agents.

In further aspects, the present invention comprises the use of such compositions, and hence of the compounds of the invention, in human and animal therapy, and methods of treatment involving such use of the compositions and compounds. The compounds of the present invention are potent and selective oxytocin receptor agonists, and so the compositions are useful in the treatment of conditions for which inadequate oxytocin-like activity is implicated in the pathophysiology. Such conditions include, but are not limited to: sexual disorders such as male erectile dysfunction, ejaculatory disorders and female sexual dysfunction, cancer of the prostate, breast, ovary and bones, osteoporosis, benign prostatic hyperplasia, post-partum bleeding, and depression. The compositions may also be used to induce labour or delivery of the placenta, to decrease arterial blood pressure, to decrease exaggerated responses to stress and to increase the nociceptive threshold.

In a preferred embodiment, the composition is used to treat male or female sexual dysfunction, and more preferably erectile dysfunction.

When used as therapeutic agents, the compositions of the present invention may be administered by any appropriate route that is known in the art. For example, they may be administered by the oral, buccal, sublingual, rectal, intravaginal, nasal, pulmonary or transdermal routes. Alternatively, they may be given by injection, including intravenous, subcutaneous and intramuscular injection. The amount given will be determined by the attending physician taking into consideration all appropriate factors. Generally a single dose will comprise between 0.1 mg and 1000 mg, preferably between 1 mg and 250 mg, of active compound. The dose may be given on a single occasion or repeatedly. When given repeatedly, it may be given at regular intervals, such as once, twice or three times daily, or on demand, according to the condition being treated.

For long-term treatment an alternative to repeated dosing may be the administration of a depot dose. For this method of administration the active agent is generally introduced into a matrix of biodegradable polymer, such as a copolymer of lactic and glycolic acids, and the formulation is given either s.c. or i.m. so as to form a deposit from which the active agent is released as the polymer degrades.

The foregoing description is further illustrated in the following examples, which are intended to demonstrate the application of the invention but not to limit the scope thereof.

### EXAMPLES

The following abbreviations have been used:
Bu butyl—alkyl residues may be further denoted as n (normal, i.e. unbranched), i (iso) and t (tertiary)
DIEA N,N-diisopropylethylamine
DMF dimethylformamide
Et ethyl
EtOAc ethyl acetate
HOBt 1-hydroxybenzotriazole
HPLC high pressure liquid chromatography
h hour(s)
Me methyl
MS mass spectrum
NMR nuclear magnetic resonance spectrum—NMR spectra were recorded in CDCl₃ unless otherwise indicated
OVA omithine vasotocin analogue
pet. ether petroleum ether boiling in the range 60-80° C.
Ph phenyl
Pn pentyl
Pr propyl
THF tetrahydrofuran
WSCD water-soluble carbodiimide (N-ethyl-N′-(3-dimethylaminopropyl)carbodiimide hydrochloride

Examples 1-9 describe the synthesis of intermediates. Compounds according to the present invention are described in Examples 10 to 134.

### Example 1

1-Benzyl-4,10-dihydropyrazolo[5,4-b][1,5]benzodiazepine 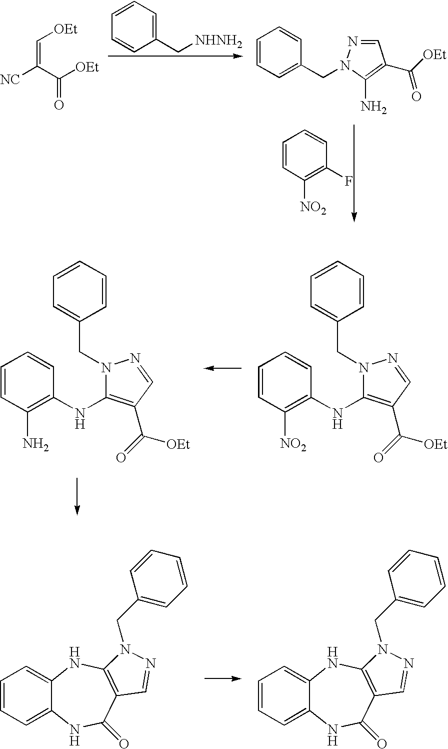
1A: Ethyl 5-amino-1-benzylpyrazole-4-carboxylate

Benzylhydrazine dihydrochloride (4.29 g, 22 mmol) was added to a solution of ethyl (ethoxymethylene)cyanoacetate (3.38 g, 20 mmol) and triethylamine (6.15 ml, 44 mmol, 2eq) in ethanol (40 ml) and the mixture was heated at reflux for 18 h. The solvent was removed in vacuo and the residue was purified by flash chromatography on silica gel (eluant 60% pet. ether/40% ethyl acetate) to yield a pale yellow solid identified as ethyl 5-amino-1-benzylpyrazole-4-carboxylate (4.3 g, 88%).

1B: Ethyl 1-benzyl-5-(2′-nitrophenylamino)pyrazole-4-carboxylate

Sodium hydride (60% dispersion in oil, 520 mg, 13 mmol) was added portionwise to a suspension of ethyl 5-amino-1-benzylpyrazole-4-carboxylate (2.2 g, 9 mmol) in anhydrous THF (30 ml) at 0° C. The mixture was allowed to warm to room temperature and stirred for 2 h then 1-fluoro-2-nitrobenzene (1.26 g, 9 mmol) was added and the resultant deep purple suspension was stirred at room temperature for 18 h. 1M KHSO₄ was added to quench the reaction and the solvent was removed in vacuo. The residue was dissolved in ethyl acetate and the solution was washed with 0.3M KHSO₄, sat. NaHCO₃ and brine, dried over Na₂SO₄ and concentrated in vacuo. The residue was purified by flash chromatography on silica gel (eluant 75% pet. ether/25% ethyl acetate) to yield ethyl 1-benzyl-5-(2′-nitrophenylamino)pyrazole-carboxylate (2.5 g, 76%).

MS [M+H]⁺366.8

1C: Ethyl 5-(2′-aminophenylamino)-1-benzylpyrazole-4-carboxylate

Ethyl 1-benzyl-5-(2′-nitrophenylamino)pyrazole-4-carboxylate (2.5 g, 6.8 mmol) was dissolved in ethyl acetate/ethanol (1:1, 100 ml) and hydrogenated over 10% Pd/C catalyst for 70 minutes. The mixture was filtered through Celite® filter agent and the filtrate was concentrated in vacuo to give a white solid identified as ethyl 5-(2′-aminophenylamino)-1-benzylpyrazole-4-carboxylate (1.5 g, 86%).

MS [M+H]⁺337.2

1D: 1-Benzyl-4,10-dihydropyrazolo[5,4-b][1,5]benzodiazepin-4(5H)-one

A solution of ethyl 5-(21-aminophenylamino)-1-benzylpyrazole-4-carboxylate (1.75 g, 5.2 mmol) in acetic acid/2-propanol (1:9, 40 ml) was heated at reflux for 3 days. The solvent was removed in vacuo and the residue was azeotroped with toluene to give an off-white solid that was purified by flash chromatography on silica gel (eluant 35% pet. ether/65% ethyl acetate) to yield a white solid identified as 1-benzyl-4,10-dihydropyrazolo[5,4-b][1,5]benzodiazepin-4(5H)-one (780 mg, 52%).

MS [M+H]⁺291.1

1E: 1-Benzyl-4,10-dihydropyrazolo[5,4-b][1,5]benzodiazepine

LiAlH₄ (365 mg, 10 mmol) was added portionwise to a suspension of 1-benzyl-4,10-dihydropyrazolo[5,4-b][1,5]benzodiazepin-4(5H)-one (780 mg, 2.7 mmol) in anhydrous THF (15 ml) at 0° C. over 10 min. The resulting suspension was heated at reflux for 18 h, then allowed to cool to room temperature. A further portion of LiAlH₄ (90 mg, 2.5 mmol) was added and the mixture was heated at refluxed for 3 h. The mixture was cooled to 0° C., 35% ammonia solution (1 ml) was added dropwise over 10 min and the mixture was stirred at room temperature for 1 h. The resulting suspension was filtered through Celite® filter agent and the filtrate was concentrated in vacuo to give a white solid identified as 1-benzyl-4,10-dihydropyrazolo[5,4-b][1,5]benzodiazepine (450 mg, 60%).

MS [M+H]⁺276.9

### Example 2

1-Methyl-4,10-dihydropyrazolo[4,5-c]pyrido[2,3-b][1,4]diazepine 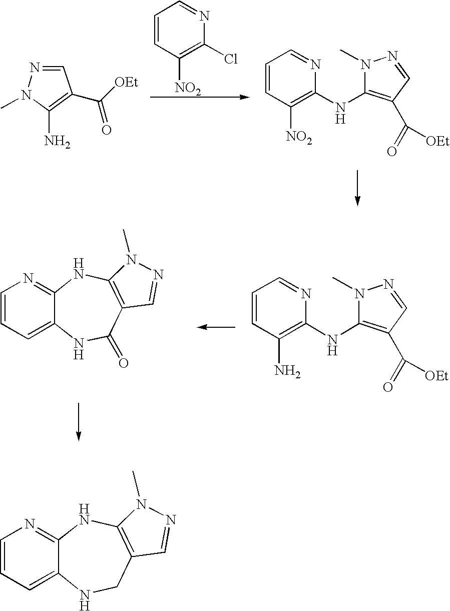
2A: Ethyl 1-methyl-2-(3′-nitro-2′-pyridylamino)pyrazole-4-carboxylate

Sodium hydride (60% dispersion in oil, 600 mg, 15 mmol) was added portionwise to a suspension of ethyl 5-amino-1-methylpyrazole-4-carboxylate (1.69 g, 10 mmol) in anhydrous THF (15 ml) at 0° C. The mixture was stirred for 2 h at room temperature then 2-chloro-3-nitropyridine (1.58 g, 10 mmol) was added and the resulting deep red suspension was stirred at room temperature for 18 h. 1M KHSO₄ was added to quench the reaction and the solvent was removed in vacuo. The residue was dissolved in ethyl acetate and the solution was washed with 0.3M KHSO₄, sat. NaHCO₃ and brine, dried over Na₂SO₄ and concentrated in vacuo. The residue was purified by flash chromatography on silica gel (eluant 30% pet. ether/70% ethyl acetate) to give ethyl 1-methyl-2-(3′-nitro-2′-pyridylamino)pyrazole-4-carboxylate (1.95 g, 67%).

MS [M+H]⁺292.0

2B: Ethyl 2-(3′-amino-2′-pyridylamino)-1-methylpyrazole-4-carboxylate

A solution of ethyl 1-methyl-2-(31-nitro-2′-pyridylamino)pyrazole-4-carboxylate (1.95 g, 6.7 mmol) in ethanol (100 ml) was hydrogenated over 10% Pd/C catalyst for 3 h. The reaction mixture was filtered through Celite® filter agent and the filtrate was concentrated in vacuo to give a white solid identified as ethyl 2-(3′-amino-2′-pyridylamino)-1-methyl-pyrazole-4-carboxylate (1.5 g, 86%).

2C: 1-Methyl-4,10-dihydropyrazolo[4,5-c]pyrido[2,3-b][1,4]diazepin-4(5H)-one

A solution of ethyl 2-(3′-amino-2′-pyridylamino)-1-methylpyrazole-4-carboxylate (1.5 g, 5.75 mmol) in acetic acid/2-propanol (1:9, 50 ml) was heated at reflux for 3 days. The solvent was removed in vacuo and the residue was azeotroped with toluene; The residue was purified by recrystallization from ethanol and then flash chromatography on silica gel (eluant 95% chloroform/4% methanol/1% acetic acid) to give a white solid identified as 1-methyl-4,10-dihydropyrazolo[4,5-c]pyrido[2,3-b][1,4]diazepin-4(5H)-one (560 mg, 45%).

2D: 1-Methyl-4,10-dihydropyrazolo[4,5-c]pyrido[2,3-b][1,4]diazepine

LiAlH4 (365 mg, 10 mmol) was added portionwise to a suspension of 1-methyl-4,10-dihydropyrazolo[4,5-c]pyrido[2,3-b][1,4]diazepin-4(5H)-one (560 mg, 2.6 mmol) in anhydrous THF (30 ml) at 0° C. over 10 minutes. The resulting suspension was heated at reflux for 18 h. The reaction was cooled to 0° C. and 35% ammonia solution (1 ml) was added dropwise over 10 minutes, then the mixture was stirred at room temperature for 1 h. The resulting suspension was filtered through Celite® filter agent and the filtrate was concentrated in vacuo to give a white solid identified as 1-methyl-4,10-dihydropyrazolo[4,5-c]pyrido[2,3-b][1,4]diazepine (410 mg, 78%).

MS [M+H]⁺202.1.

### Example 3

tert-Butyl 4-aminomethyl-3-chlorobenzoate 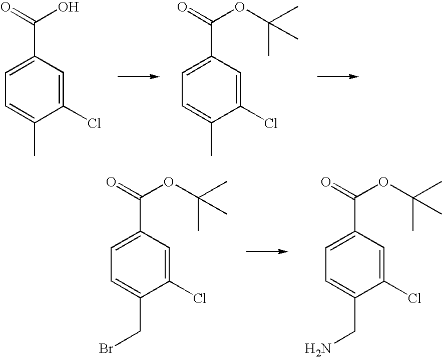
3A: tert-Butyl 3-chloro-4-methylbenzoate
Thionyl chloride (11 ml, 150 mmol) was added to a suspension of 3-chloro-4-methyl-benzoic acid (5.12 g, 30 mmol) in toluene (25 ml) and the mixture was heated at reflux for 2 h. The solvent was removed in vacuo and the residue was azeotroped with toluene three times, then dissolved in anhydrous THF (40 ml) and cooled to 0° C. Lithium tert-butoxide (2.4 g, 30 mmol) was added and the mixture was stirred at room temperature for 3 days. Water (5 ml) was added and the solvent was removed in vacuo. The residue was dissolved in ethyl acetate. The solution was washed with 0.3M KHSO₄, sat. NaHCO₃ and brine, dried over Na₂SO₄ and concentrated in vacuo to give a pale yellow gum identified as tert-butyl 3-chloro-4-methylbenzoate (5.4 g, 79%).
3B: tert-Butyl 4-bromomethyl-3-chlorobenzoate

N-Bromosuccinimide (4.27 g, 24 mmol) and 2,2′-azo-bis(2-methylpropionitrile) (394 mg, 2.4 mmol) were added to a solution of tert-butyl 3-chloro-4-methylbenzoate (5.4 g, 23.8 mmol) in carbon tetrachloride (75 ml) and the mixture was heated at reflux for 18 h. The solvent was removed in vacuo and the residue was purified by flash chromatography on silica gel (eluant 95% pet.ether/5% ethyl acetate) to give a white solid identified as tert-butyl 4-bromomethyl-3-chlorobenzoate (5.7 g, 78%).

3C: tert-Butyl 4-aminomethyl-3-chlorobenzoate

Ethanol (100 ml) was saturated with ammonia, then tert-butyl 4-bromomethyl-3-chloro-benzoate (5.7 g, 18.7 mmol) was added and the mixture was stirred at room temperature for 2 h. The solvent was removed in vacuo and the residue was triturated with diethyl ether to give a white solid identified as tert-butyl 4-aminomethyl-3-chlorobenzoate (4.1 g, 91%).

### Example 4

4-(tert-Butyloxycarbonylaminomethyl)-3-chlorobenzoic acid 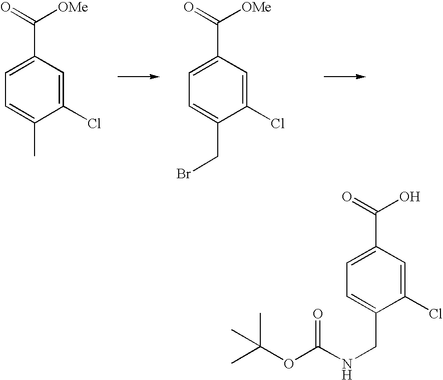
4A. Methyl 4-bromomethyl-3-chlorobenzoate

To a solution of methyl 3-chloro-4-methylbenzoate (5.0 g, 27.1 mmol) in carbon tetrachloride (50 ml) were added N-bromosuccinimide (5.8 g, 32.0 mmol) and 2,2′-azo-bis(2-methylpropionitrile) (0.442 g, 2.70 mmol). The mixture was heated at reflux for 18 h, then allowed to cool to room temperature and concentrated in vacuo. The residue was purified by flash chromatography on silica (eluant pet. ether 5% ethyl acetate/95% pet. ether) to give an oil identified as methyl 4-bromomethyl-3-chlorobenzoate (5.96 g, 84%).

4B. 4-(4(tert-Butyloxycarbonylaminomethyl)-3-chlorobenzoic acid

To a saturated solution of ammonia in ethanol (170 ml) was added methyl 4-bromomethyl-3-chlorobenzoate from Example 4A (5.5 g, 20.9 mmol). The mixture was stirred at room temperature for 1 h and then concentrated in vacuo. The residue was triturated with diethyl ether and the resultant white crystals were filtered off and washed with more diethyl ether. To a solution of this solid in water (100 ml) were added solutions of di-tert-butyl dicarbonate (5.0 g, 23.0 mmol) in dioxan (100 ml) and sodium hydroxide (1.86 g, 46.0 mmol) in water (100 ml). The mixture was stirred at room temperature for 18 h and then concentrated in vacuo. The aqueous residue was acidified with citric acid and extracted with chloroform/2-propanol. The organic layer was washed with water, dried over MgSO₄, and concentrated in vacuo to give a white solid identified as 4-(tert-butyloxy-carbonylaminomethyl)-3-chlorobenzoic acid (2.8 g, 67%).

### Example 5

4-(tert-Butyloxycarbonylaminomethyl)-3-nitrobenzoic acid 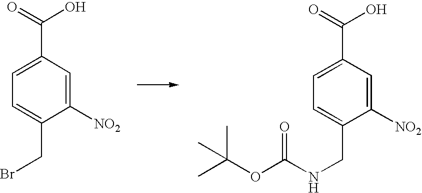
4-Bromomethyl-3-nitrobenzoic acid (4.75 g, 18.2 mmol) was reacted following the method of Example 4B to give a yellow solid identified as 4-(tert-butyloxycarbonylaminomethyl)-3-nitrobenzoic acid (2.6 g, 49%).

### Example 6

4-Cyano-3-methylbenzoic acid

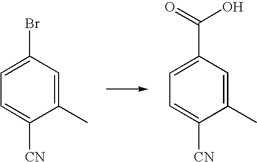

To a solution of 4-bromo-2-methylbenzonitrile (2.0 g, 10.2 mmol) in THF (100 ml) at −78° C. under a nitrogen atmosphere was added dropwise a 2.5M solution of n-butyl lithium (4.48 m[, 11.2 mmol). The mixture was stirred at −78° C. for 1 h and then poured onto solid carbon dioxide (5 g) in THF (50 ml). The mixture was allowed to warm to room temperature. Water was added (200 ml) and the mixture was extracted with diethyl ether (3 times). The aqueous layer was acidified by addition of concentrated HCl and extracted with chloroform (3 times). The combined chloroform extracts were washed with water, dried over MgSO₄, and concentrated in vacuo to give a white solid identified as 4-cyano-3-methylbenzoic acid (1.2 g, 73%).

### Example 7

4-Cyano-2-methylbenzoic acid

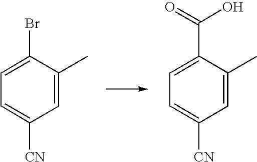

4-Bromo-3-methylbenzonitrile (2.0 g, 10.2 mmol) was reacted following the method of Example 6. The product was triturated with hexane to give a yellow solid identified as 4-cyano-2-methylbenzoic acid (0.96 g, 59%).

### Example 8

4-(tert-Butyloxycarbonylaminomethyl)-2-fluorobenzoic acid 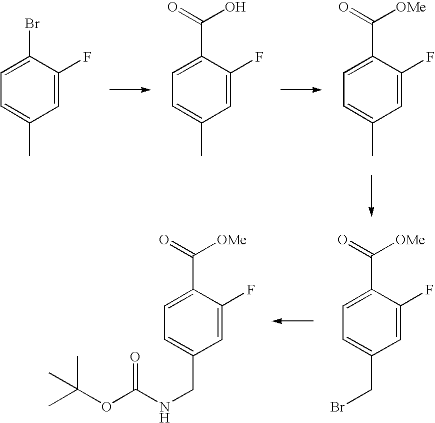
8A. 2-Fluoro-4-methylbenzoic acid
4-Bromo-3-fluorotoluene (8.33 g, 44.07 mmol) was reacted following the method of Example 6 to give a white solid identified as 2-fluoro-4-methylbenzoic acid (4.89 g, 72%).
8B. Methyl 2-fluoro-4-methylbenzoate

To a solution of 2-fluoro-4-methylbenzoic acid (6.04 g, 39.18 mmol) in toluene (80 ml) was added thionyl chloride (65 ml, 89.11 mmol). The mixture was heated at reflux for 2.5 h, cooled and concentrated in vacuo. The residue was dissolved in dichloromethane (50 ml) and methanol (50 ml) was added. The mixture was stirred at room temperature for 2.5 h and then concentrated in vacuo. The residue was dissolved in dichloromethane (100 ml), washed with saturated sodium bicarbonate solution and brine, dried over MgSO₄, and concentrated in vacuo to give a tan solid identified as methyl 2-fluoro-4-methylbenzoate (5.07 g, 77%).

8C. Methyl 4-bromomethyl-2-fluorobenzoate

Methyl 2-fluoro-4-methylbenzoate (5.07 g, 30.16 mmol) was reacted following the method of Example of 4A. The product was purified by flash chromatography on silica (eluant 20% ethyl acetate/80% pet. ether) to give an oil identified as methyl 4-bromomethyl-2-fluorobenzoate (5.9 g, 80%).

8D. 4-(tert-Butyloxycarbonylaminomethyl)-2-fluorobenzoic acid

Methyl 4-bromomethyl-2-fluorobenzoate (5.9 g, 24.13 mmol) was reacted following the method of Example 4B. The product was recrystallised from dioxan/pet. ether to give white crystals identified as 4-(tert-butyloxycarbonylaminomethyl)-2-fluorobenzoic acid (2.46 g, 38%).

### Example 9

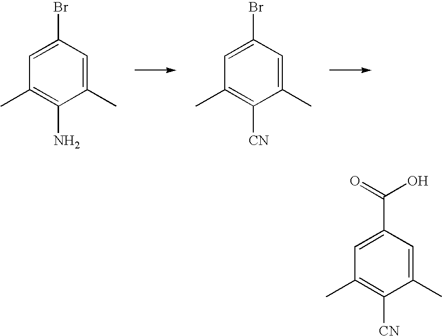 9A. 4-Bromo-2,6-dimethylbenzonitrile
4-Bromo-2,6-dimethylaniline (4.49 g, 22.4 mmol) was taken up in water (25 ml) and concentrated hydrochloric acid (8.0 ml) was added. The mixture was sonicated to form a fine suspension and then cooled to 0° C. A solution of sodium nitrite (1.67 g, 24.2 mmol) in water (5 ml) was then added dropwise so as to maintain the temperature of the reaction between 0-5° C. The mixture was stirred at 0-5° C. for 30 minutes and then neutralised by addition of solid sodium bicarbonate. The resulting solution was then added portionwise to a solution of copper cyanide (2.42 g, 27.0 mmol) and potassium cyanide (3.65 g, 56.1 mmol) in water (25 ml) at 70° C. The mixture was stirred at 70° C. for 30 minutes, allowed to cool and then extracted with toluene (2 times). The combined extracts were washed with water and brine, dried over MgSO₄, and concentrated in vacuo. The residue was purified by flash chromatography on silica (eluant 5% ethyl acetate/95% pet. ether) to give an orange solid identified as 4-bromo-2,6-dimethylbenzonitrile (3.2 g, 68%).
9B. 4-Cyano-3,5-dimethylbenzoic acid

4-Bromo-2,6-dimethylbenzonitrile (3.20 g, 15.2 mmol) was reacted following the method of Example 6 to give a tan solid identified as 4-cyano-3,5-dimethylbenzoic acid (1.5 g, 56%).

### Example 10

443-Methyl 4-(piperazine-1-carbonylaminomethyl)benzoyl)-5,6,7,8-tetrahydrothieno[3,2-b]azepine hydrochloride 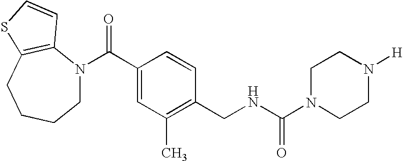
10A: 4-(3-Methyl-4-cyanobenzoyl)-5,6,7,8-tetrahydrothieno[3,2-b]azepine

Thionyl chloride (5 ml, 68.55 mmol) was added to a stirred suspension of 4 cyano-3-methylbenzoic acid (1.43 g, 8.90 mmol) in dichloromethane (20 ml). The mixture was heated at reflux for 2 h, cooled to room temperature and concentrated in vacuo. The residue was azeotroped with dichloromethane then dissolved in dichloromethane 20 ml. The resulting solution was slowly added to a stirred solution of 5,6,7,8-tetrahydrothieno[3,2-b]azepine (1.36 g, 8.90 mmol) and triethylamine (3.70 ml, 26.54 mmol) in dichloromethane (30 ml). The mixture was stirred at room temperature for 24 h, washed with 1M KHSO₄, saturated NaHCO₃ and brine, then concentrated in vacuo. The residue was purified by flash chromatography on silica gel (eluant 25% EtOAc/pet. ether) to give a brown solid identified as 4-(3-methyl-4-cyanobenzoyl)-5,6,7,8-tetrahydrothieno[3,2-b]azepine (1.70 g, 71%).

10B: 4-(4-Aminomethyl-3-methylbenzoyl)-5,6,7,8-tetrahydrothieno[3,2-b]azepine

Cobalt(II) chloride hexahydrate (2.84 g, 11.94 mmol) was added to a solution of 4-(3-methyl-4-cyanobenzoyl)-5,6,7,8-tetrahydrothieno[3,2-b]azepine (1.709, 5.70 mmol) in methanol (70 ml) at 0° C. Sodium borohydride (2.22 g, 58.68 mmol) was added portionwise at 0° C. and the mixture was stirred at 0° C. for 30 min then at room temperature for 2 h. Saturated ammonium chloride was then added and the mixture was stirred for 30 min then concentrated in vacuo. The residue was azeotroped with toluene then extracted with chloroform. The extracts were washed with brine and concentrated in vacuo to give a white solid identified as 4-(4-aminomethyl-3-methylbenzoyl)-5,6,7,8-tetrahydrothieno[3,2-b]azepine (1.129, 65%).

10C: 4-(4-(4-(tert-Butyloxycarbonyl)piperazine-1-carbonylaminomethyl)-3-methyl-benzoyl)-5,6,7,8-tetrahydrothieno[3,2-b]azepine

1,1′-Carbonyldiimidazole (234 mg, 1.45 mmol) was added to a solution of 4-(4-amino-methyl-3-methylbenzoyl)-5,6,7,8-tetrahydrothieno[3,2-b]azepine (400 mg, 1.33 mmol) and DIEA (0.3 ml, 1.72 mmol) in DMF (20 ml) and the mixture was stirred at room temperature for 30 min. tert-Butyl piperazine-1-carboxylate (281 mg, 1.50 mmol) was added and the mixture was stirred at room temperature for 24 h then concentrated in vacuo. The residue was taken up in chloroform and the solution was washed with 1M KHSO₄ and brine, then concentrated in vacuo. The residue was purified by flash chromatography on silica gel (eluant 75% EtOAc/pet. ether) to give a white solid identified as 4-(4-(4-(tert-butyloxycarbonyl)piperazine-1-carbonylaminomethyl)-3-methylbenzoyl)-5,6,7,8-tetrahydrothieno[3,2-b]azepine (588 mg, 86%).

10D: 443-Methyl-4-(piperazine-1-carbonylaminomethyl)benzoyl)-5,6,7,8-tetrahydrothieno[3,2-b]azepine hydrochloride

A solution of 4-(4-(4-(tert-butyloxycarbonyl)piperazine-1-carbonylaminomethyl)-3-methyl-benzoyl)-5,6,7,8-tetrahydrothieno[3,2-b]azepine (588 mg, 1.15 mmol) in 4N HCl/dioxan (10 ml) was stirred at room temperature for 30 min then concentrated in vacuo. The residue was dissolved in acetonitrile/water and lyophilised to give a white solid identified as 4-(3-methyl-4-(piperazine-1-carbonylaminomethyl)benzoyl)-5,6,7,8-tetrahydrothieno-[3,2-b]azepine hydrochloride(393 mg, 76%).

¹H NMR: d₆-DMSO δ 1.60-1.74 (2H, m), 1.82-1.94 (2H, m), 2.17 (3H, s), 2.86-2.95 (2H, m), 2.96-3.10 (4H, m), 3.35-3.45 (2H, m), 3.50-3.64 (4H, m), 4.16 (2H, s), 6.26 (1H, br s), 6.85-7.10 (4H, m), 7.24 (1H, brs), 9.28 (1H, brs) ppm.

MS: [M+H]⁺⁼413.2

### Example 11

5-(4-(4-Cyclopropylmethylpiperazine-1-carbonylaminomethyl)-3-methylbenzoyl)-1-methyl-4,10-dihydropyrazolo[5,4-b][1,5]benzodiazepine 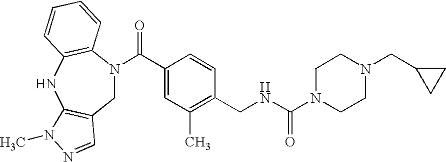
11A: 5-(4-Cyano-3-methylbenzoyl)-1-methyl-4,10-dihydropyrazolo[5,4-b[1,51-benzodiazepine

Thionyl chloride (1.8 ml, 27 mmol) was added to a stirred suspension of 4-cyano-3-methyl-benzoic acid (1.29 g, 8.0 mmol) in toluene (25 ml). The mixture was heated at reflux for 2 hr, cooled to room temperature and concentrated in vacuo. The residue was azeotroped with toluene then dissolved in dichloromethane (10 ml). The resulting solution was added to a stirred suspension of 1-methyl-4,10-dihydropyrazolo[5,4-b][1,5]benzodiazepine (1.6 g, 8 mmol) and triethylamine (1.4 ml, 10 mmol) in dichloromethane (15 ml). The mixture was stirred overnight at room temperature then concentrated in vacuo. The residue was partitioned between chloroform and 0.3M KHSO₄. The aqueous phase was extracted with chloroform/2-propanol (80:20). The combined organic phases were washed with sat. NaHCO₃ and brine, dried over Na₂SO₄ and concentrated in vacuo. The residue was purified by flash chromatography on silica gel (eluant 5% methanol/chloroform) to give a pale yellow solid identified as 5-(4-cyano-3-methylbenzoyl)-1-methyl-4,10-dihydropyrazolo[5,4-b][1,5]benzodiazepine (2.4 g, 87%).

11B: 5-(4-Aminomethyl-3-methylbenzoyl)-1-methyl-4,10-dihydropyrazolo[5,4-b][1,51]-benzodiazepine

Cobalt(II) chloride hexahydrate (1.59 g, 6.7 mmol) was added to an ice-cold solution of 5-(4-cyano-3-methylbenzoyl)-1-methyl-4,10-dihydropyrazolo[5,4-b][1,5]benzodiazepine (1.15 g, 3.35 mmol) in methanol (35 ml). Sodium borohydride (1.27 g, 33.5 mmol) was added portionwise at 0° C. and the mixture was stirred at RT for 1 hr, then quenched with 1M KHSO₄ and concentrated in vacuo. The aqueous residue was diluted with 1M KHSO₄ (40 ml) and filtered through Celite® filter agent. The filtrate was washed with diethyl ether (2×50 ml) then basified with 2M NaOH and extracted with chloroform. The organic phase was dried over Na₂SO₄ and concentrated in vacuo to give a pale brown solid identified as 5-(4-aminomethyl-3-methylbenzoyl)-1-methyl-4,10-dihydropyrazolo[5,4-b][1,5]benzodiazepine (745 mg, 64%).

11C: 5-(4-(4-(tert-Butyloxycarbonyl)piperazine-1-carbonylaminomethyl)-3-methyl-benzoyl)-1-methyl-4,10-dihydropyrazolo[5,4-b][1,5]benzodiazepine

1,1′-Carbonyldiimidazole (76 mg, 0.47 mmol) was added to a solution of 5-(4-(aminomethyl)-3-methylbenzoyl)-1-methyl-4,10-dihydropyrazolo[5,4-b][1,5]benzo-diazepine (150 mg, 0.43 mmol) and DIEA (0.1 ml, 0.57 mmol) in DMF (10 ml). The solution was stirred for 30 min, tert-butyl piperazine-1-carboxylate (91 mg, 0.49 mmol) was added and stirring was continued for 72 h. The mixture was concentrated in vacuo and the residue was taken up in chloroform. The solution was washed with water and brine, dried and concentrated in vacuo. The residue was purified by flash chromatography on silica gel (eluant 100% EtOAc then 10% methanol/EtOAc) to give a white solid identified as 5-(4-(4-(tert-butyloxycarbonyl)piperazine-1-carbonylaminomethyl)-3-methylbenzoyl)-1-methyl-4,10-dihydropyrazolo[5,4-b][1,5]benzodiazepine (160 mg, 66%).

11 D: 1-Methyl-5-(3-methyl-4-piperazine-1-carbonylaminomethyl)-benzoyl)4,10-dihydropyrazolo[5,4-b][1,5]benzodiazepine hydrochloride

A solution of 5-(4-(4-(tert-butyloxycarbonyl)piperazine-1-carbonylaminomethyl)-3-methyl-benzoyl)-1-methyl-4,10-dihydropyrazolo[5,4-b][1,5]benzodiazepine (160 mg, 0.29 mmol) in 4N HCl/dioxan (15 ml) was stirred at room temperature for 30 min then concentrated in vacuo. The residue was azeotroped with diethyl ether to give a white solid identified as 1-methyl-5-(3-methyl-4-(piperazine-1-carbonylaminomethyl)-benzoyl)-4,10-dihydropyrazolo[5,4-b][1,5]benzodiazepine hydrochloride (130 mg, 90%).

11E: 5-(4-(4-Cyclopropylmethylpiperazine-1-carbonylaminomethyl)-3-methyl-benzoyl)-1-methyl-4,10-dihydropyrazolo[5,4-b][1,5]benzodiazepine

To a solution of 1-methyl-5-(3-methyl-4-(piperazine-1-carbonylaminomethyl)-benzoyl)-4,10-dihydropyrazolo[5,4-b][1,5]benzodiazepine hydrochloride (100 mg, 0.20 mmol) and triethylamine (0.5 ml, 3.59 mmol) in THF (10 ml) were added cyclopropanecarboxaldehyde (14 mg, 0.20 mmol) and sodium cyanoborohydride (15 mg, 0.24 mmol) and the resulting mixture was stirred at room temperature for 24 h then concentrated in vacuo. The residue was dissolved in ethyl acetate and the resulting solution was washed with saturated NaHCO₃, water and brine, dried and concentrated in vacuo. The residue was purified by flash chromatography on silica gel (eluant 10% methanol/EtOAc) to give a white solid identified as 5-(4-(4-cyclopropylmethylpiperazine-1-carbonylaminomethyl)-3-methylbenzoyl)-1-methyl-4,10-dihydropyrazolo[5,4-b][1,5]benzodiazepine (35 mg, 35%).

¹H NMR: d₄-MeOH δ 0.14 (2H, q, J=4.7 Hz), 0.51-0.59 (2H, m), 0.82-0.95 (1H, m), 2.15 (3H, s), 2.28 (2H, d, J=6.7 Hz), 2.52 (4H, t, J=4.9 Hz), 3.43 (4H, t, J=4.9 Hz), 3.80 (3H, s), 3.95 (1H, d, J=14.4 Hz), 4.23 (2H, s), 5.78 (1H, d, J=14.6 Hz), 6.61-6.74 (2H, m), 6.99 (2H, s), 7.03 (1H, s), 7.057.14 (1H, m), 7.19-7.24 (2H, m) ppm.

MS: [M+H]⁺=514.3

### Example 12

5-(4-(4-Benzylpiperazine-1-carbonylaminomethyl)-3-methylbenzoyl)-1-methyl-4,10-dihydropyrazolo[5,4-b][1,5]benzodiazepine

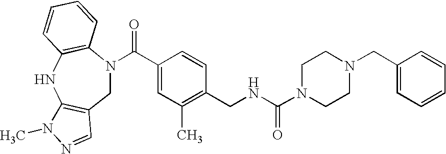

To a solution of 1-methyl-5-(3-methyl-4-(piperazine-1-carbonylaminomethyl)-benzoyl)-4,10-dihydropyrazolo[5,4-b][1,5]benzodiazepine hydrochloride (100 mg, 0.20 mmol) and triethylamine (0.5 ml, 3.59 mmol) in THF (10 ml) were added benzaldehyde (21 mg, 0.20 mmol) and sodium cyanoborohydride (15 mg, 0.24 mmol) and the resulting mixture was stirred at room temperature for 24 h then concentrated in vacuo. The residue was dissolved in ethyl acetate and the resulting solution was washed with saturated NaHCO₃, water and brine, dried and concentrated in vacuo. The residue was purified by flash chromatography on silica gel (eluant 5% methanol/EtOAc) to give a white solid identified as 5-(4-(4-benzylpiperazine-1-carbonylaminomethyl)-3-methylbenzoyl)-1-methyl-4,10-dihydropyrazolo[5,4-b][1,5]benzodiazepine (37 mg, 34%).

¹H NMR: δ 2.10 (3H, s), 2.36-2.48 (4H, m), 3.29-3.44 (4H, m), 3.48-3.51 (2H, m), 3.76 (3H, s), 3.96 (1H, d, J=14.6 Hz), 4.224.28 (2H, m), 4.614.68 (1H, m), 5.88 (1H, d, J=14.6 Hz), 6.46 (1H, s,) 6.62-6.74 (2H, m), 6.82-6.96 (3H, m), 6.98-7.11 (2H, m), 7.19-7.34 (5H, m) ppm.

MS: (M+H]⁺=550.2

### Example 13

5-(4-(4-(3-Hydroxybenzyl)piperazine-1-carbonylaminomethyl)-3-methylbenzoyl)-1-methyl-4,10-dihydropyrazolo[5,4-b][1,5]benzodiazepine 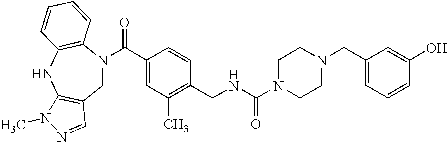
13A: 3-(tert-Butyldimethylsilyloxy)toluene

tert-Butyldimethylsilyl chloride (3.00 g, 22.00 mmol) was added to a solution of m-cresol (2.00 g, 18.00 mmol) and triethylamine (4 ml, 28.7 mmol) in dichloromethane (50 ml) at 0° C. The mixture was stirred at room temperature for 24 h then concentrated in vacuo. The residue was purified by flash chromatography on silica gel (eluant 10% EtOAc/pet. ether) to give a colourless oil identified as 3-(tert-butyldimethylsilyloxy)toluene (3.60 g, 88%).

13B: 3-(tert-Butyldimethylsilyloxy)benzyl bromide N-Bromosuccinimide (2.90 g, 16.20 mmol) and AIBN (266 mg, 1.62 mmol) were added to a stirred solution of 3-(tert-butyldimethylsilyloxy)toluene (3.60 g, 16.20 mmol) in carbon tetrachloride (120 ml) and the mixture was heated at reflux for 24 h, then allowed to cool to room temperature and concentrated in vacuo. The residue was purified by flash chromatography on silica gel (eluant cyclohexane) to give a colourless oil identified as 3-(tert-butyldimethylsilyloxy)benzyl bromide (2.45 g, 50%).

13C: tert-Butyl 4-(3-hydroxybenzyl)piperazine-1-carboxylate

Sodium hydride (406 mg, 60% dispersion in oil, 10.15 mmol) was added portionwise to a stirred solution of tert-butyl piperazine-1-carboxylate in DMF (50 ml) at 0° C. The mixture was allowed to warm to room temperature over 1 h, then a solution of 3-(tert-butyldimethylsilyloxy)benzyl bromide (2.44 g, 8.10 mmol) in DMF (10 ml) was added dropwise and the mixture was stirred at room temperature for 24 h. Water was added and the mixture was stirred for 30 min then poured into EtOAc. The organic phase was washed with saturated NaHCO₃ and brine, then concentrated in vacuo. The residue was purified by flash chromatography on silica gel (eluant 40% EtOAc/pet. ether) to give a light brown oil identified as tert-butyl 4-(3-hydroxybenzyl)piperazine-1-carboxylate (2.00 g, 84%).

13D: 1-(3-Hydroxybenzyl)piperazine dihydrochloride

A solution of tert-butyl 4-(3-hydroxybenzyl)piperazine-1-carboxylate (1.94 g, 6.60 mmol) in 4N HCl/dioxan (10 ml) was stirred at room temperature for 30 min then concentrated in vacuo. The residue was triturated with diethyl ether to give a white solid identified as 1-(3-hydroxybenzyl)piperazine dihydrochloride (1.10 g, 63%).

13E: 5-(4-(4-(3-Hydroxybenzyl)piperazine-1-carbonylaminomethyl)-3-methyl-benzoyl)-1-methyl-4,10-dihydropyrazolo[5,4-b][1,5]benzodiazepine

1,1′-Carbonyldiimidazole (15 mg, 0.09 mmol) was added to a stirred solution of 5-(4-(aminomethyl)-3-methylbenzoyl)-1-methyl-4,10-dihydropyrazolo[5,4-b][1,5]benzo-diazepine (31 mg, 0.09 mmol) and DIEA (0.1 ml 0.57 mmol) in DMF (5 ml). The solution was stirred for 1 h, 1-(3-hydroxybenzyl)piperazine dihydrochloride (27 mg, 0.10 mmol) was added and stirring was continued at room temperature for 24 h. The mixture was concentrated in vacuo and the residue was taken up in EtOAc. The solution was washed with saturated NaHCO₃ and brine, then concentrated in vacuo. The residue was purified by flash chromatography on silica gel (eluant 20% methanol/EtOAc) to give a white solid identified as 5-(4-(4-(3-hydroxybenzyl)piperazine-1-carbonylaminomethyl)-3-methylbenzoyl)-1-methyl-4,10-dihydropyrazolo[5,4-b][1,5]benzodiazepine (45 mg, 90%).

¹H NMR: δ 2.15 (3H, s), 2.41 (4H, t, J=4.7 Hz), 3.40 (4H, t, J=4.7 Hz), 3.46 (2H, s), 3.80 (3H, s), 3.97 (1H, d, J=14.6 Hz), 4.22 (2H, s), 4.90 (1H, m), 5.78 (1H, d, J=14.6 Hz), 6.62-6.79 (5H, m), 6.99 (2H, s), 7.03-7.27 (6H, m) ppm.

MS: [M+H]⁺=566.1

### Example 14

5-(4-(4-(3-Hydroxymethylbenzyl)piperazine-1-carbonylaminomethyl)-3-methyl-benzoyl)-1-methyl-4,10-dihydropyrazolo[6,4-b][1,5]benzodiazepine 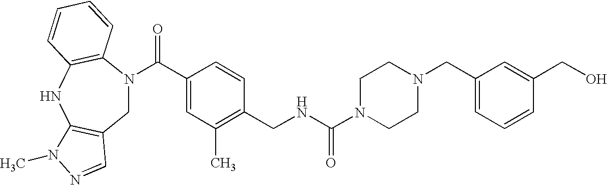
14A: tert-Butyl 4-(3-(methyloxycarbonyl)benzyl)piperazine-1-carboxylate

Methyl 3-(bromomethylbenzoate) (1.23 g, 5.37 mmol) was added to a stirred solution of tert-butyl piperazine-1-carboxylate (1.00 g, 5.37 mmol) and triethylamine (1.50 ml, 10.74 mmol) in dichloromethane (20 ml). The solution was stirred at room temperature for 24 h then concentrated in vacuo. The residue was purified by flash chromatography on silica gel (eluant EtOAc) to give a white solid identified as tert-butyl 4-(3-(methyloxycarbonyl)benzyl)piperazine-1-carboxylate (1.55 g, 86%).

14B: tert-Butyl 4-(3-carboxybenzyl)piperazine-1-carboxylate

Lithium hydroxide monohydrate (339 mg, 9.27 mmol) was added to a solution of tert-butyl 4-(3-(methyloxycarbonyl)benzyl)piperazine-1-carboxylate (1.55 g, 4.63 mmol) in THF (10 ml) and water (2 ml). The solution was stirred at room temperature for 24 h then acidified to pH 5 with 0.3M KHSO₄ and extracted successively with chloroform and dichloromethane. The combined extracts were concentrated in vacuo to give a white solid identified as tert-butyl 4-(3-carboxybenzyl)piperazine-1-carboxylate (1.09 g, 74%).

14C: tert-Butyl 4-(3-(hydroxymethyl)benzyl)piperazine-1-carboxylate

Isobutyl chloroformate (0.47 ml, 3.64 mmol) was slowly added to an ice-cold solution of tert-butyl 4-(3-carboxybenzyl)piperazine-1-carboxylate (1.06 g, 3.31 mmol) and N-methylmorpholine (0.80 ml, 7.28 mmol) in THF (15 ml). The solution was stirred at 0° C. for 45 min and then filtered. The filtrate was added to an ice-cold solution of sodium borohydride (313 mg, 8.27 mmol) in water (10 ml). The stirred mixture was allowed to warm to room temperature over 2 h and then concentrated in vacuo. The residue was taken up in EtOAc and the solution was washed with water and brine then concentrated in vacuo. The residue was purified by flash chromatography on silica gel (eluant.EtOAc) to give a white solid identified as tert-butyl 4-(3-(hydroxymethyl)benzyl)piperazine-1-carboxylate (230 mg, 23%).

14D: 1-(3-(Hydroxymethyl)benzyl)piperazine dihydrochloride

A solution of tert-butyl 4-(3-(hydroxymethyl)benzyl)piperazine-1-carboxylate (230 mg, 0.75 mmol) in 4N HCl/dioxan (10 ml) was stirred at room temperature for 45 min then concentrated in vacuo. The residue was azeotroped with toluene to give a white solid identified as 1-(3-(hydroxymethyl)benzyl)piperazine dihydrochloride (158 mg, 75%).

14E: 5-(4-(4-(3-Hydroxymethylbenzyl)piperazine-1-carbonylaminomethyl)-3-methyl-benzoyl)-1-methyl-4,10-dihydropyrazolo[5,4-b][1,5]benzodiazepine

1,1′-Carbonyldiimidazole (20 mg, 0.12 mmol) was added to a solution of 5-(4-(aminomethyl)-3-methylbenzoyl)-1-methyl-4,10-dihydropyrazolo[5,4-b][1,5]benzo-diazepine (35 mg, 0.10 mmol) in DMF (3 ml). The solution was stirred for 1 h, a solution of 1-(3-(hydroxymethyl)benzyl)piperazine dihydrochloride (31 mg, 0.11 mmol) and DIEA (54 μl, 0.30 mmol) in DMF (2 ml) was added and the mixture was stirred at room temperature for 24 h then concentrated in vacuo. The residue taken up in chloroform and the solution was washed with brine and concentrated in vacuo. The residue was purified by flash chromatography on silica gel (eluant 7% methanol/chloroform) to give a white solid identified as 5-(4-(4-(3-hydroxymethylbenzyl)piperazine-1-carbonylaminomethyl)-3-methylbenzoyl)-1-methyl-4,10-dihydropyrazolo[5,4-b][1,5]benzodiazepine (27 mg, 50%).

¹H NMR: δ 2.00 (3H, s), 2.32-2.36 (4H, m), 3.32-3.45 (4H, m), 3.46 (2H, s), 3.63 (3H, s), 3.91 (1H, d, J=14.6 Hz), 4.104.20 (1H, m), 4.66 (2H, s), 5.28-5.29 (1H, m), 5.80 (1H, d, J=14.3 Hz), 6.50-7.30 (15H, m) ppm.

MS: [M+H]⁺=580.3

### Example 15

1-Methyl-5-(3-methyl-4-(4-(4-picolyl)piperazine-1-carbonylaminomethyl)benzoyl)-4,10-dihydropyrazolo[5,4-b][1,5]benzodiazepine

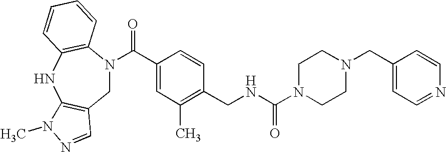

To a solution of 1-methyl-5-(3-methyl-4-(piperazine-1-carbonylaminomethyl)-benzoyl)-4,10-dihydropyrazolo[5,4-b][1,5]benzodiazepine hydrochloride (100 mg, 0.20 mmol) and triethylamine (0.5 ml, 3.59 mmol) in THF (10 ml) were added 4-pyridinecarboxaldehyde (21 mg, 0.20 mmol) and sodium cyanoborohydride (15 mg, 0.24 mmol) and the resulting mixture was stirred at room temperature for 24 h then concentrated in vacuo. The residue was dissolved in ethyl acetate and the resulting solution was washed with saturated NaHCO₃, water and brine, dried and concentrated in vacuo. The residue was purified by flash chromatography on silica gel (eluant 10%-30% methanol/EtOAc) to give a white solid identified as 1-methyl-5-(3-methyl-4-(4-(4-picolyl)piperazine-1-carbonylaminomethyl)benzoyl)-4,10-dihydropyrazolo[5,4-b][1,5]benzodiazepine (33 mg, 30%).

¹H NMR: δ 2.13 (3H, s), 2.342.49 (4H, m), 3.29-3.47 (4H, m), 3.76 (3H, s), 3.96 (1H, d, J=14.8 Hz), 4.254.27 (2H, d, J=4.7 Hz), 4.50-4.60 (1H, m), 5.90 (1H, d, J=14.4 Hz), 6.25 (1H, s), 6.63-6.71 (2H, m), 6.84 (2H, s), 6.92 (1H, s), 7.00-7.12 (2H, m), 7.25 (5H, s), 8.53 (2H, d, J=5.9 Hz) ppm.

MS: [M+H]⁺=551.1

### Example 16

5-(4-(4-(2-Hydroxyethyl)piperazine-1-carbonylaminomethyl)-3-methylbenzoyl)-1-methyl-4,10-dihydropyrazolo[5,4-b][1,5]benzodiazepine

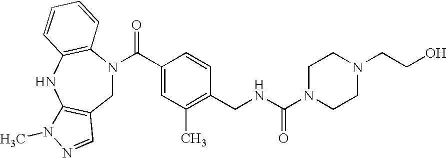

1,1′-Carbonyldiimidazole (20 mg, 0.19 mmol) was added to a solution of 5-(4-(aminomethyl)-3-methylbenzoyl)-1-methyl-4,10-dihydropyrazolo[5,4-b][1,5]benzo-diazepine (31 mg, 0.09 mmol) in DMF (3 ml). The solution was stirred at room temperature for 1 h, a solution of 1-(2-hydroxyethyl)piperazine (13 mg, 0.10 mmol) in DMF (2 ml) was added and stirring was continued for 72 h. The solution was concentrated in vacuo and the residue was partitioned between chloroform and brine. The organic layer was separated and concentrated in vacuo. The residue was purified by flash chromatography on silica gel (eluant 7% methanol/chloroform) to give a white solid identified as 5-(4-(4-(2-hydroxyethyl)piperazine-1-carbonylaminomethyl)-3-methyl-benzoyl)-1-methyl-4,10-dihydropyrazolo[5,4-b][1,5]benzodiazepine (22 mg, 48%).

¹H NMR: δ 2.09 (3H, s), 2.42-2.59 (6H, m), 2.91-3.01 (1H, m), 3.33-3.62 (6H, m), 3.67 (3H, s), 3.93-3.98 (1H, m), 4.20-4.23 (2H, m), 5.00-5.03 (1H, m), 5.84-5.90 (1H, m), 6.64-7.25 (9H, m) ppm.

MS: [M+H]⁺=504.2

### Example 17

1-Methyl-5-(3-methyl-4-(4-(3-(methylthio)propyl)piperazine-1-carbonylaminomethyl)-benzoyl)-4,10-dihydropyrazolo[5,4-b][1,5]benzodiazepine

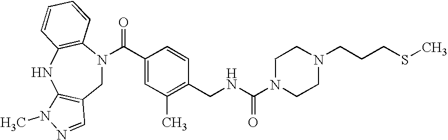

To a solution of 1-methyl-5-(3-methyl-4-(piperazine-1-carbonylaminomethyl)-benzoyl)-4,10-dihydropyrazolo[5,4-b][1,5]benzodiazepine hydrochloride (100 mg, 0.20 mmol) and triethylamine (0.5 ml, 3.59 mmol) in THF (10 ml) were added 3-(methylthio)-propionaldehyde (21 mg, 0.20 mmol) and sodium cyanoborohydride (15 mg, 0.24 mmol) and the resulting mixture was stirred at room temperature for 24 h then concentrated in vacuo. The residue was dissolved in ethyl acetate and the resulting solution was washed with saturated NaHCO₃, water and brine, dried and concentrated in vacuo. The residue was purified by flash chromatography on silica gel (eluant 20% methanol/EtOAc) to give a white solid identified as 1-methyl-5-(3-methyl-4-(4-(3-(methylthio)-propyl)piperazine-1-carbonylaminomethyl)benzoyl)-4,10-dihydropyrazolo[5,4-b][1,5]-benzodiazepine (41 mg, 38%).

¹H NMR: δ 1.63-1.80 (3H, m), 2.04-2.12 (4H, m), 2.33-2.42 (6H, m), 2.48 (2H, t, J=6.7 Hz), 3.29-3.39 (4H, m), 3.71 (3H, s), 3.93 (1H, d, J=14.4 Hz), 4.124.30 (2H, m), 4.57-4.70 (1H, m), 5.85 (1H, d, J=14.6 Hz), 6.44 (1H, s), 6.59-6.71 (2H, m), 6.83-6.88 (2H, m), 6.92-7.08 (2H, m), 7.14-7.27 (2H, m) ppm.

MS: [M+H]⁺=548.0

### Example 18

5-(4-(4-(2-Aminoethyl)piperazine-1-carbonylaminomethyl)-3-methylbenzoyl)-1-methyl-4,10-dihydropyrazolo[5,4-b][1,5]benzodiazepine dihydrochloride 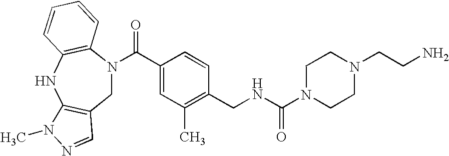
18A: Benzyl 442-hydroxyethyl)piperazine-1-carboxylate Benzyl chloroformate (3.40 ml, 24.00 mmol) was slowly added to an ice-cold stirred solution of 1-(2-hydroxyethyl)piperazine (2.60 g, 20.00 mmol) and DIEA (7.0 ml, 40.0 mmol) in dichloromethane (75 ml). The mixture was allowed to warm to room temperature and stirred for 24 h then concentrated in vacuo. The residue was purified by flash chromatography on silica gel (eluant 6% methanol/chloroform) to give a colourless gum identified as benzyl 4-(2-hydroxyethyl)piperazine-1-carboxylate (4.80 g, 91%).
18B: Benzyl 4-(2-bromoethyl)piperazine-1-carboxylate

Carbon tetrabromide (7.23 g, 21.80 mmol) was added to an ice-cold stirred solution of benzyl 4-(2-hydroxyethyl)piperazine-1-carboxylate (4.80 g, 18.20 mmol) in dichloromethane (50 ml). The solution was stirred for 5 min, triphenylphosphine (5.95 g, 22.70 mmol) was added, and the mixture was allowed to warm to room temperature and stirred for 3 h. Silica gel was added and the solvent was removed in vacuo. The residue was purified by flash chromatography on silica gel (eluant 50% EtOAc/pet. ether) to give a colourless gum identified as benzyl 4-(2-bromoethyl)piperazine-1-carboxylate (3.45 g, 58%).

18C: Benzyl 4-(2-(tert-butyloxycarbonylamino)ethyl)piperazine-1-carboxylate

Benzyl 4-(2-bromoethyl)piperazine-1-carboxylate (3.45 g, 10.55 mmol) was added to an ice-cold saturated solution of ammonia in ethanol (60 ml). The mixture was allowed to warm to room temperature and stirred for 4 h, then concentrated in vacuo. The residue was triturated with diethyl ether. The resultant solid was suspended in dichloromethane (75 ml) and triethylamine (2.25 ml, 16.00 mmol). The suspension was cooled to 0° C. and di-tert-butyl dicarbonate (2.40 g, 11.00 mmol) was added. The mixture was allowed to warm to room temperature and stirred for 24 h then concentrated in vacuo. The residue was taken up in EtOAc. The solution was washed with saturated NaHCO₃ and brine, then concentrated in vacuo. The residue was purified by flash chromatography on silica gel (eluant 3% methanol/chloroform) to give a yellow gum identified as benzyl 4-(2-(tert-butyloxycarbonylamino)ethyl)piperazine-1-carboxylate (2.60 g, 68%).

18D: tert-Butyl 2-(1-piperazinyl)ethylcarbamate

Hydrogen was passed through a degassed solution of benzyl 4-(2-(tert-butyloxycarbonylamino)ethyl)piperazine-1-carboxylate (2.60 g, 7.16 mmol) in methanol (50 ml) containing 10% palladium on carbon (500 mg) for 2 h. The reaction mixture was filtered through Celite® and the filtrate was concentrated in vacuo to give a yellow gum identified as tert-butyl 2-(1-piperazinyl)ethylcarbamate (1.60 g, 97%).

18E: 5-(4-(4-(2-(tert-Butyloxycarbonylaminoethyl)piperazine-1-carbonylamino-methyl)-3-methylbenzoyl)-1-methyl-4,10-dihydropyrazolo[5,4-b][1,5]benzodiazepine

1,1′-Carbonyldiimidazole (25 mg, 0.15 mmol) was added to a solution of 5-(4-(aminomethyl)-3-methylbenzoyl)-1-methyl-4,10-dihydropyrazolo[5,4-b][1,5]benzo-diazepine (31 mg, 0.09 mmol) and DIEA (0.1 ml, 0.57 mmol) in DMF (5 ml). The solution was stirred for 1 h, tert-butyl 2-(1-piperazinyl)ethylcarbamate (22 mg, 0.10 mmol) was added and stirring was continued at room temperature for 24 h. The mixture was concentrated in vacuo and the residue was taken up in EtOAc. The solution was washed with saturated NaHCO₃ and brine, then concentrated in vacuo. The residue was purified by flash chromatography on silica gel (eluant 20% methanol/EtOAc) to give a white solid identified as 5-(4-(4-(2-(tert-butyloxycarbonylaminoethyl)piperazine-1-carbonylaminomethyl)-3-methylbenzoyl)-1-methyl-4,10-dihydropyrazolo[5,4-b][1,5]-benzodiazepine (44 mg, 81%).

18F: 5-(4-(4-(2-Aminoethyl)piperazine-1-carbonylaminomethyl)-3-methylbenzoyl)-1-methyl-4,10-dihydropyrazolo[5,4-b][1,5]benzodiazepine dihydrochloride

A solution of 5-(4-(4-(2-(tert-butyloxycarbonylaminoethyl)piperazine-1 Carbonylamino-methyl)-3-methylbenzoyl)-1-methyl-4,10-dihydropyrazolo[5,4-b][1,5]benzodiazepine (42 mg, 0.07 mmol) in 4N HCl/dioxan (5 ml) was stirred at room temperature for 30 min then concentrated in vacuo. The residue was dissolved in acetonitrile/water and lyophilised to give a white solid identified as 5-(4-(4-(2-aminoethyl)piperazine-1-carbonylaminomethyl)-3-methylbenzoyl)-1-methyl-4,10-dihydropyrazolo[5,4-b][1,5]benzodiazepine dihydrochloride (37 mg, 92%).

¹H NMR: δ 2.17 (3H, s), 3.30-3.35 (4H, m), 3.41-3.50 (1H, m), 3.56-3.72 (4H, m), 4.00 (3H, s), 4.04 (1H, s), 4.26 (2H, s), 4.83-4.89 (2H, m), 5.88 (1H, d, J=15 Hz), 6.83-6.84 (2H, m), 6.92-7.13 (4H, m), 7.15-7.28 (1H, m), 7.36 (1H, d, J=7.9 Hz), 7.96 (1H, s) ppm.

MS: [M+H]⁺=503.5

### Example 19

1-Methyl-5-(3-methyl-4-(4-methylperhydro-1,4-diazepine-1-carbonylaminomethyl)benzoyl)-4,10-dihydropyrazolo[5,4-b][1,5]benzodiazepine

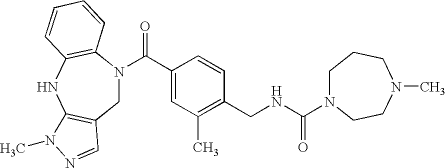

1,1′-Carbonyldiimidazole (37 mg, 0.23 mmol) was added to a solution of 5-(4-(aminomethyl)-3-methylbenzoyl)-1-methyl-4,10-dihydropyrazolo[5,4-b](1,5]benzo-diazepine (75 mg, 0.22 mmol) in DMF (2 ml). The solution was stirred for 1 h, a solution of 1-methylhomopiperazine (27 mg, 0.24 mmol) and DIEA (31 mg, 0.24 mmol) in DMF (1 ml) was added and stirring was continued for 24 h. The mixture was concentrated in vacuo and the residue was purified by chromatography on silica gel (eluant 30/2/1-1/1/1 chloroform/methanol/concentrated ammonia) to give a white solid identified as 1-methyl-5-(3-methyl-4-(4-methylperhydro-1,4-diazepine-1-carbonylaminomethyl)benzoyl)4,10-dihydropyrazolo[5,4-b][1,5]benzodiazepine (38 mg, 36%).

¹H NMR: δ 1.80-1.99 (2H, m), 2.10 (3H, s), 2.35 (3H, s), 2.51-2.69 (4H, m), 3.39 (2H, t, J=5.9 Hz), 3.45-3.68 (2H, m), 3.63 (3H, s), 3.95 (1H, d, J=14.6 Hz), 4.23 (2H, t, J=4.2 Hz), 4.65-4.75 (1H, m), 5.85 (1H, d, J=14.6 Hz), 6.65-6.75 (2H, m), 6.76-6.88 (2H, m), 6.90-7.09 (2H, m), 7.11-7.22 (2H, m) ppm.

MS: [M+H]+=488.2

### Example 20

5-(4-(4-(2-Hydroxyethyl)piperazine-1-carbonylaminomethyl)-3-methylbenzoyl)-1-methyl-4,10-dihydropyrazolo[4,5-c]pyrido[2,3-b][1,4]diazepine 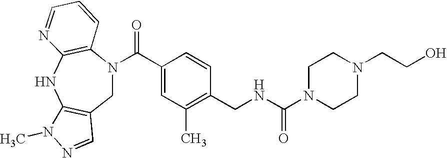
20A: 5-(4-Cyano-3-methylbenzoyl)-1-methyl-4,10-dihydropyrazolo[4,5-c]pyrido[2,3-b][1,4]diazepine
Thionyl chloride (0.6 ml, 9.00 mmol) was added to a suspension of 4-cyano-3-methylbenzoic acid (322 mg, 2.00 mmol) in toluene (10 ml). The mixture was heated at reflux for 2 h, allowed to cool and concentrated in vacuo. The residue was azeotroped with toluene and then taken up in dichloromethane (5 ml). The solution was added slowly to a stirred solution of 1-methyl-4,10-dihydropyrazolo[4,5-c]pyrido[2,3-b][1,4]diazepine (400 mg, 2.00 mmol) and triethylamine (0.35 ml, 2.50 mmol) in dichloromethane (5 ml). The mixture was stirred at room temperature for 24 h then concentrated in vacuo. The residue was purified by flash chromatography on silica gel (eluant 5% methanol/chloroform) to give an orange solid identified as 5-(4-cyano-3-methylbenzoyl)-1-methyl-4,10-dihydropyrazolo[4,5-c]pyrido[2,3-b][1,4]diazepine (500 mg, 73%).
20B: 5-(4-Aminomethyl-3-methylbenzoyl)-1-methyl-4,10-dihydropyrazolo[4,5-c]pyrido[2,3-b][1,4]diazepine

Cobalt(II) chloride hexahydrate (690 mg, 2.90 mmol) was added to an ice-cold stirred solution of 5-(4-cyano-3-methylbenzoyl)-1-methyl-4,10-dihydropyrazolo[4,5-c]pyrido[2,3-b][1,4]diazepine (500 mg, 1.45 mmol) in methanol (15 ml). Sodium borohydride (570 mg, 15.00 mmol) was added portionwise and the mixture was stirred at room temperature for 1 h. 1M KHSO₄ was added, the methanol was removed in vacuo, and the aqueous residue was filtered through Celite®. The filtrate was washed with diethyl ether, basified to pH12 with 2M sodium hydroxide and extracted with chloroform. The chloroform extracts were washed with brine and concentrated in vacuo to give a pale orange solid identified as 5-(4-aminomethyl-3-methylbenzoyl)-1-methyl-4,10-dihydropyrazolo[4,5-c]pyrido[2,3-b][1,4]diazepine (400 mg, 79%).

20C: 5-(4-(4-(2-Hydroxyethyl)piperazine-1-carbonylaminomethyl)-3-methylbenzoyl)-1-methyl-4,10-dihydropyrazolo[4,5-c]pyrido[2,3-b][1,4]diazepine

1,1′-Carbonyldiimidazole (20 mg, 0.12 mmol) was added to a solution of 5-(4-aminomethyl-3-methylbenzoyl)-1-methyl-4,10-dihydropyrazolo[4,5-c]pyrido[2,3-b](1,4]-diazepine (35 mg, 0.10 mmol) in DMF (3 ml). The solution was stirred for 1 h, a solution of 1-(2-hydroxyethyl)piperazine (13 mg, 0.10 mmol) and DIEA (18 μl, 0.10 mmol) in DMF (2 ml) was added and the mixture was stirred at room temperature for 24 h then concentrated in vacuo. The residue taken up in chloroform and the solution was washed with brine and concentrated in vacuo. The residue was purified by flash chromatography on silica gel (eluant 7% methanol/chloroform) to give a pale yellow solid identified as 5-(4-(4-(2-hydroxyethyl)piperazine-1-carbonylaminomethyl)-3-methylbenzoyl)-1-methyl-4,10-dihydropyrazolo[4,5-c]pyrido[2,3-b][1,4]diazepine (29 mg, 58%).

¹H NMR: δ 2.42 (3H, br s), 2.44-2.60 (7H, m), 3.20-3.40 (4H, m), 3.55-3.65 (2H, m), 3.79 (3H, s), 3.85-4.00 (1H, m), 4.26 (2H, br s), 4.88 (1H, br s), 5.80-5.95 (1H, m), 6.60 (1H, br s), 6.80-7.30 (6H, m), 8.00 (1H, s) ppm.

MS: [M+H]⁺=505.2

### Examples 21-134

The Following Compounds were Prepared Using Analogous Methods to Those Described

### Examples 21-30

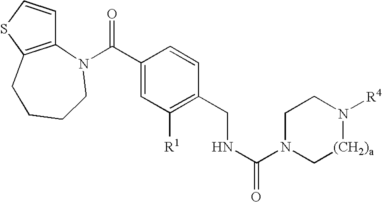

| | a | R¹ | R⁴ | MS: [M + H]⁺ |
|---|---|---|---|---|
| 21 | 1 | Cl | CH₃ | 447.3 |
| 22 | 2 | Cl | CH₃ | 461.3 |
| 23 | 1 | Me | CH₂CH₂CH₂CH₃ | 469.3 |
| 24 | 1 | Me | CH₂CH(CH₃)CH₂CH₃ | 483.3 |
| 25 | 1 | Me | 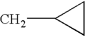 | 467.3 |
| 26 | 1 | Cl | 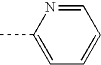 | 510.3 |
| 27 | 1 | Me | 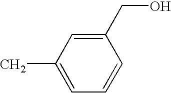 | 533.3 |
| 28 | 1 | Me | 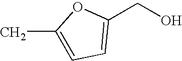 | 523.2 |
| 29 | 1 | Me | 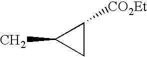 | 539.3 |
| 30 | 1 | Me | CH₂CH₂OH | 457.2 |

### Examples 31-46

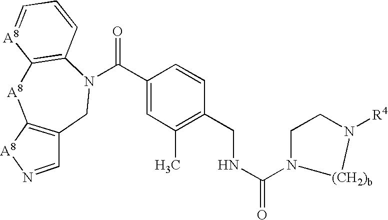

| | A³ | A⁶ | A⁸ | b | R⁴ | MS: [M + H]⁺ |
|---|---|---|---|---|---|---|
| 31 | OH | NH | N—Me | 3 | H | 474.1 |
| 32 | CH | NH | N—Me | 3 | CH₂CH(CH₃)CH₂CH₃ | 544.3 |
| 33 | CH | NH | N—Me | 3 | CH₂C(CH₃)₃ | 544.3 |
| 34 35 | CH CH | NH N—Me | N—Me N—Me | 3 1 | 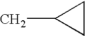 | 528.3 514.3 |
| 36 | N | NH | N—Me | 2 | 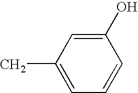 | 680.2 |
| 37 | OH | N—Me | N—Me | 2 | 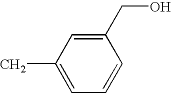 | 594.3 |
| 38 39 | CH CH | NH NH | N—Me N—Me | 3 2 | 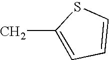 | 570.3 556.3 |
| 40 41 | CH CH | NH NH | N—Me N—Me | 3 2 | 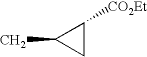 | 600.3 586.3 |
| 42 | N | NH | N—Me | 2 | CH₂CH₂NH₂ | 504.1 |
| 43 | N | NH | N—Me | 2 | CH₂CH₂CH₂NH₂ | 518.3 |
| 44 45 | CH N | NH NH | N—Me N—Me | 2 2 | 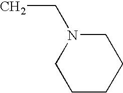 | 571.4 572.3 |
| 46 | CH | NH | N—CH₂Ph | 2 | CH₂CH₂OH | 580.2 |

### Examples 47-117

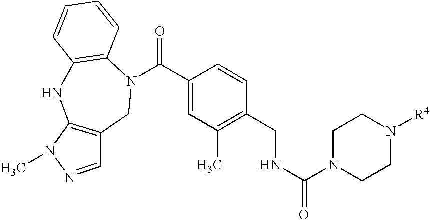

| | R⁴ | MS: [M + H]⁺ |
|---|---|---|
| 47 | H | 460.2 |
| 48 | CH₃ | 474.2 |
| 49 | CH₂CH₃ | 488.2 |
| 50 | CH₂CH₂CH₃ | 502.3 |
| 51 | CH₂CH₂CH₂CH₃ | 516.3 |
| 52 | CH₂CH₂CH₂CH₂CH₃ | 530.3 |
| 53 | CH₂CH₂CH₂CH₂CH₂CH₃ | 544.3 |
| 54 | CH₂CH₂CH(CH₃)₂ | 530.3 |
| 55 | CH₂CH(CH₃)CH₂CH₃ | 530.3 |
| 56 | CH₂CH(CH₂CH₃)₂ | 544.3 |
| 57 | CH₂CH₂C(CH₃)₃ | 544.2 |
| 58 | 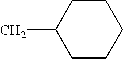 | 556.3 |
| 59 | CH₂CH═CH₂ | 500.1 |
| 60 | 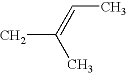 | 528.3 |
| 61 | 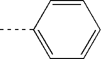 | 536.2 |
| 62 | 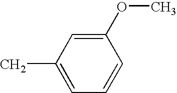 | 580.3 |
| 63 | 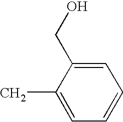 | 580.3 |
| 64 | 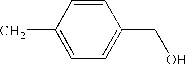 | 580.2 |
| 65 | 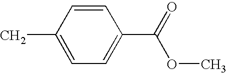 | 608.3 |
| 66 | 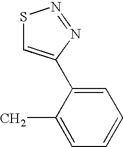 | 634.2 |
| 67 | 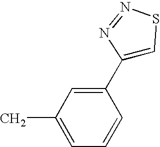 | 634.2 |
| 68 | 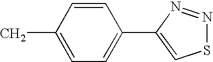 | 634.2 |
| 69 | 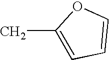 | 540.3 |
| 70 | 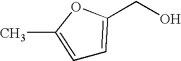 | 570.2 |
| 71 | 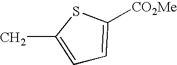 | 614.2 |
| 72 | 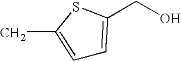 | 586.3 |
| 73 | 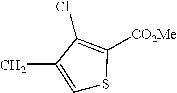 | 648.2 |
| 74 | 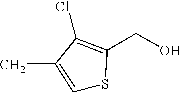 | 620.2 |
| 75 | 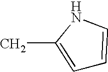 | 538.2 |
| 76 | 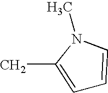 | 553.1 |
| 77 | 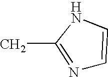 | 540.2 |
| 78 | 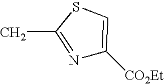 | 629.2 |
| 79 | 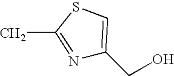 | 587.3 |
| 80 | 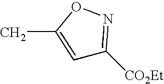 | 599.2 |
| 81 | 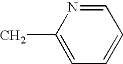 | 551.3 |
| 82 | 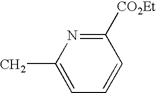 | 609.1 |
| 83 | 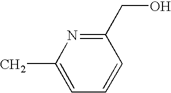 | 581.3 |
| 84 | 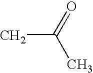 | 516.3 |
| 85 | 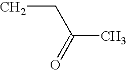 | 530.2 |
| 86 | 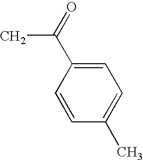 | 592.2 |
| 87 | CH₂CH₂CO₂CH₃ | 546.3 |
| 88 | CH₂CH₂CO₂H | 532.1 |
| 89 | CH₂CH₂CH₂CO₂CH₃ | 560.2 |
| 90 | CH₂CH₂CN | 513.4 |
| 91 | CH₂CH₂N₃ | 529.2 |
| 92 | 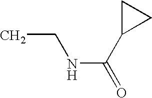 | 571.2 |
| 93 | 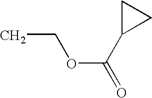 | 572.2 |
| 94 | 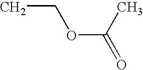 | 546.3 |
| 95 | 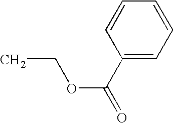 | 608.3 |
| 96 | 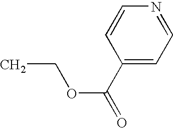 | 609.3 |
| 97 | 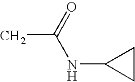 | 557.3 |
| 98 | 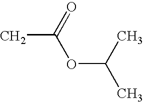 | 560.3 |
| 99 | 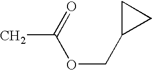 | 572.2 |
| 100 | CH₂CH₂CH₂NH₂ | 517.3 |
| 101 | CH₂CH₂N(CH₂CH₃)₂ | 559.3 |
| 102 | 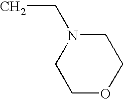 | 573.2 |
| 103 | 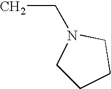 | 557.3 |
| 104 | CH₂CH₂CH₂CH₂OH | 532.3 |
| 105 | 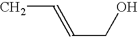 | 530.2 |
| 106 | 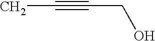 | 536.9 [M + Na]⁺ |
| 107 | 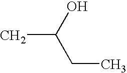 | 532.3 |
| 108 | 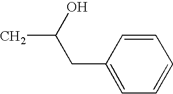 | 594.3 |
| 109 | 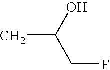 | 536.2 |
| 110 | 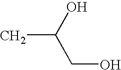 | 534.3 |
| 111 | 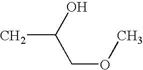 | 548.1 |
| 112 | 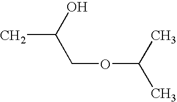 | 576.3 |
| 113 | CH₂CH₂OCH₃ | 518.2 |
| 114 | CH₂CH₂OCH₂CH₃ | 532.3 |
| 115 | CH₂CH₂OCH₂CH₂OCH₃ | 562.3 |
| 116 | CH₂CH₂OCH₂CH₂OCH₂CH₃ | 576.3 |
| 117 | 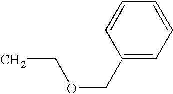 | 594.3 |
| 130 | 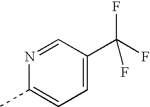 | 605.7 |

### Examples 118-120

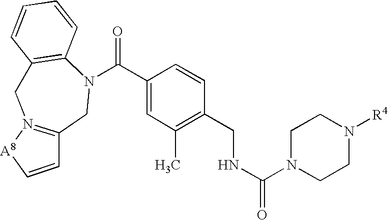

| | A⁸ | R⁴ | MS: [M + H]⁺ |
|---|---|---|---|
| 118 | CH | 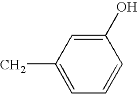 | 550.3 |
| 119 | CH | 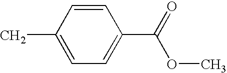 | 592.3 |
| 120 | N | CH₂CH₂OH | 489.0 |

### Examples 121-128

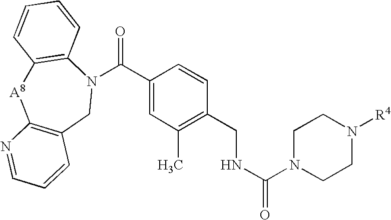

| | A⁸ | R⁴ | MS: [M + H]⁺ |
|---|---|---|---|
| 121 | NH | 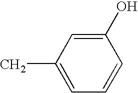 | 584.9 [M + Na]⁺ |
| 122 | NH | 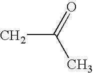 | 513.3 |
| 123 | NH | CH₂CH₂CH₂NH₂ | 514.3 |
| 124 | NH | CH₂CH₂NH₂ | 500.3 |
| 125 | O | CH₂CH₂OH | 502.1 |
| 126 | NH | CH₂CH₂OH | 501.3 |
| 127 | NH | 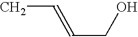 | |
| 128 | NH | 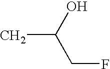 | 533.3 |

### Examples 129

4-Cyclopropylmethyl-piperazine-1-carboxylic acid 2-methyl-4-(5,6,7,8-tetrahydro-ine-4-carbonyl)-benzyl ester 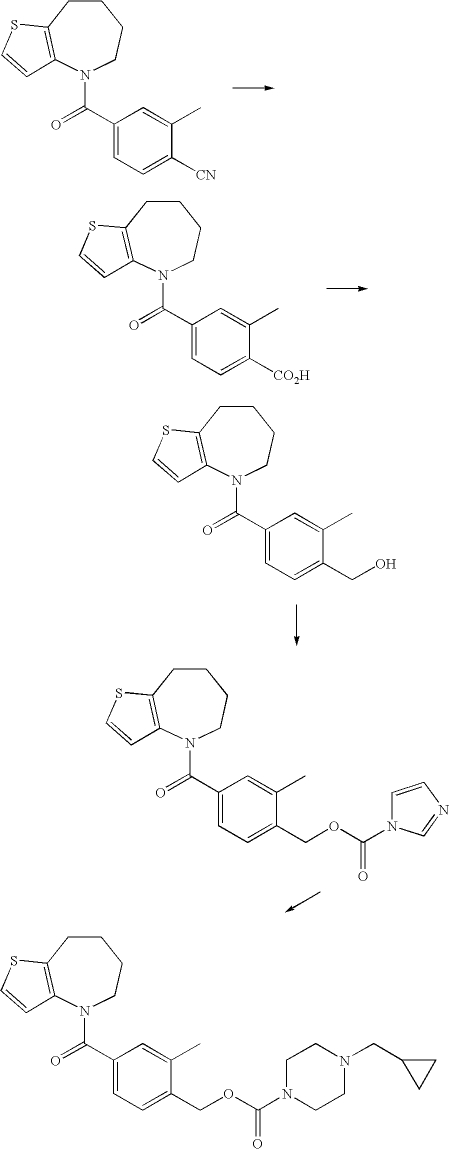
4-(4-Carboxy-3-methylbenzoyl)-5,6,7,8-tetrahydrothieno[3,2-b]azepine

A suspension of 4-(4-cyano-3-methylbenzoyl)-5,6,7,8-tetrahydrothieno[3,2-b]azepine (1 g, 3.3 mmol) in conc. sulphuric acid/water (1:1, 30 ml) was heated at reflux for 5 hr. The resulting solution was cooled to RT, diluted with water (20 ml) and extracted with chloroform (3×20 ml). The combined organic phases were extracted with sat. NaHCO₃ (2×20 ml). The combined aqueous extracts were acidified with 1M KHSO₄ and extracted with chloroform (3×20 ml). These chloroform extracts were combined, washed with brine, dried over Na₂SO₄ and concentrated in vacuo to give a pale brown solid identified as 4-(4-carboxy-3-methylbenzoyl)-5,6,7,8-tetrahydrothieno[3,2-b]azepine. (225 mg, 23%).

4-(4-Hydroxymethyl-3-methylbenzoyl)-5,6,7,8-tetrahydrothieno[3,2-b]azepine

Isobutyl chloroformate (250 μl, 2 mmol) was added to a solution of 4-(4-carboxy-3-methylbenzoyl)-5,6,7,8-tetrahydrothieno[3,2-b]azepine (470 mg, 1.48 mmol) and N-methylmorpholine (230 μl, 2.1 mmol) in THF (15 ml) at 0° C. and the mixture was stirred for 1 hr. The resultant suspension was filtered and the filtrate was added to a solution of sodium borohydride (131 mg, 3.45 mmol) in water (15 ml) at 0° C. The solution was stirred at RT for 2 hr, then sat. NH₄Cl (5 ml) was added and the THF was removed in vacuo. The remaining solution was diluted with water and extracted with chloroform (3×20 ml). The combined organic phases were washed with brine, dried over Na₂SO₄ and concentrated in vacuo to give a pale brown solid identified as 4-(4-hydroxymethyl-3-methylbenzoyl)-5,6,7,8-tetrahydrothieno[3,2-b]azepine (330 mg, 74%).

4-(4-(1-Imidazolecarbonyloxymethyl)-3-methylbenzoyl)-,6,7,8-tetrahydrothieno[3,2-b]azepine

1,1′-Carbonyldiimidazole (36 mg, 0.22 mmol) was added to a solution of 4-(4-hydroxymethyl-3-methylbenzoyl)-5,6,7,8-tetrahydrothieno[3,2-b]azepine (60 mg, 0.17 mmol) in DMF (2 ml) under nitrogen gas and the solution was stirred at RT for 18 hr. The solvent was removed in vacuo and the residue was purified by flash chromatography on silica gel (eluant 97% chloroform/3% methanol) to give a colourless gum identified as 4-(4-(1-imidazolecarbonyloxymethyl)-3-methylbenzoyl)-5,6,7,8-tetrahydrothieno[3,2-b]azepine (60 mg, 45%).

4-Cyclopropylmethyl-piperazine-1-carboxylic acid 2-methyl-4-(5,6,7,8-tetrahydro-thieno[3,2-b]azepine-4-carbonyl)-benzyl ester

A mixture of 4-(4-(1-imidazolecarbonyloxymethyl)-3-methylbenzoyl)-5,6,7,8-tetrahydrothieno[3,2-b]azepine (1.0eq), 1-cyclopropylmethyl-piperazine (1.0 eq) and DIEA (1.05eg) was heated at reflux for 48 h. The mixture was concentrated in vacuo and purified by flash chromatography on silica gel (eluant methanol/chloroform).

MS: [M+H]⁺=468

### Examples 131-132

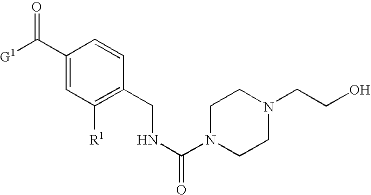

| | G¹ | R¹ | MS: [M + H]⁺ |
|---|---|---|---|
| 131 | 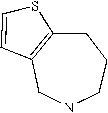 | Cl | 477.6 |
| 132 | 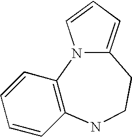 | Me | 488.7 |

### Examples 133 and 134

Prepared by analogous Methods.

### Example 133

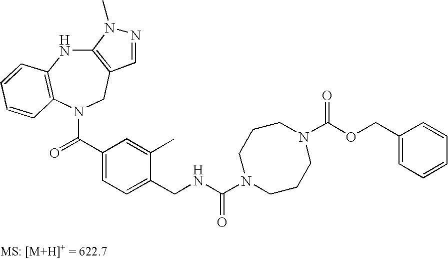

### Example 134

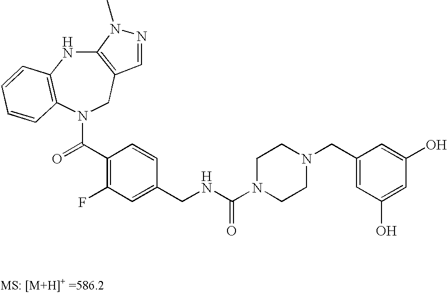

### Example 135

In vitro Testing Compounds were assayed to determine their ability to mimic the cellular consequences of OT stimulation on intact cells. In the assay, the compounds of the invention cause significant cellular activation at concentrations of 30 μM or less. Preferred compounds cause significant activation at concentrations of 300 nM or less and can induce the same maximal effect as OT. The preferred compounds are either significantly less active or completely devoid of activity in assays for vasopressin-like activity.

### Example 136

In Vivo Testing

Representative compounds were tested for activity in the rat uterine contractility model, which is a recognised test for OT agonism. The compounds increased the strength and frequency of the uterine contractions at doses below 50 mg/kg. Selected compounds were then given either i.c.v. or iv. to male rats and the erectile response was determined.

### Example 137

Tablet for Oral Administration

Tablets containing 100 mg of the compound of Example 11 as the active agent are prepared from the following:

| | | |
|---|---|---|
| Compound of Example 11 | 200.0 | g |
| Corn starch | 71.0 | g |
| Hydroxypropylcellulose | 18.0 | g |
| Carboxymethylcellulose calcium | 13.0 | g |
| Magnesium stearate | 3.0 | g |
| Lactose | 195.0 | g |
| Total | 500.0 | g |

The materials are blended and then pressed to give 2000 tablets of 250 mg, each containing 100 mg of the compound of Example 11.

The foregoing demonstrates that the compounds according to the present invention act as agonists at the oxytocin receptor and accordingly they may find utility as pharmaceutical agents for the treatment of conditions such as sexual disorders including male erectile dysfunction, ejaculatory disorders and female sexual dysfunction, cancer of the prostate, breast, ovary and bones, osteoporosis, benign prostatic hyperplasia, post-partum bleeding, and depression. The compounds may also be used to induce labour or delivery of the placenta, to decrease arterial blood pressure, to decrease exaggerated responses to stress and to increase the nociceptive threshold.

The scope of the present invention is further defined in the following claims.

1. A compound according to general formula 1, or a pharmaceutically acceptable salt thereof 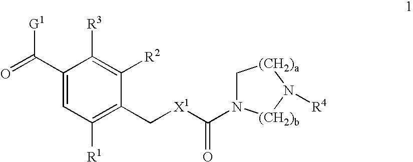
wherein:
G¹ is selected from a group according to general formula 2, a group according to general formula 3, a group according to general formula 4, a group according to general formula 5, a group according to general formula 6 and a group according to general formula 7; 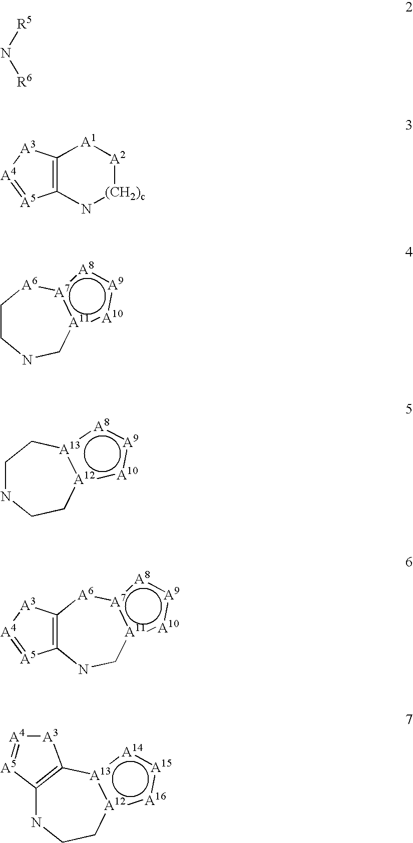
A¹ is selected from CH₂)CH(OH), NH, N-alkyl, O and S;
A² is selected from CH₂, CH(OH), C(O) and NH;
A³ is selected from S, NH, N-alkyl, —CH═CH— and —CH═N—;
A⁴ and A⁵ are each selected from CH and N;
A⁶ is selected from CH₂, NH, N-alkyl and 0;
A⁷ and A¹¹ are selected from C and N;
A⁸ and A⁹ are selected from CH, N, NH, N(CH₂)_{d}R⁷ and S;
A¹⁰ is selected from —CH═CH—, CH, N, NH, N(CH₂)_{d}R⁷ and S;
A¹² and A¹³ are selected from N and C;
A¹⁴, A¹⁵ and A¹⁶ are selected from NH, N—CH₃, S, N and CH;
X¹ is selected from O and NH;
R¹, R² and R³ are each selected from H, alkyl, O-alkyl, F, Cl and Br;
R⁴ is selected from H, alkyl, alkenyl, alkynyl, optionally substituted phenyl, optionally substituted thienyl, optionally substituted furyl, optionally substituted pyridyl,
(CO)—O— (CH₂)ₑR⁸, —(CH₂)ₑR⁸, —CH₂—CH═CH—CH₂R⁸, —CH₂—C≡C—CH₂R⁸, —(CH₂)₉—CH(OH)—(CH₂)ₕ—R⁸, —(CH₂)ᵢ—O—(CH₂)ᵢ—R⁸ and 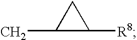
R⁵ and R⁶ are independently selected from alkyl, Ar and —(CH₂)_{f}—Ar;
R⁷ is selected from H, alkyl, optionally substituted phenyl, F, OH, O-alkyl, O-acyl, S-alkyl, NH₂, NH-alkyl, N(alkyl)₂, NH-acyl, N(alkyl)-acyl, CO₂H, CO₂-alkyl, CONH₂, CONH-alkyl, CON(alkyl)₂, CN, CF₃, optionally substituted pyridyl, optionally substituted thienyl and optionally substituted furyl;
R⁸ is selected from H, alkyl, alkenyl, alkynyl, acyl, optionally substituted phenyl, optionally substituted pyridyl, optionally substituted thienyl, optionally substituted furyl, optionally substituted pyrollyl, optionally substituted pyrazolyl, optionally substituted imidazolyl, optionally substituted oxazolyl, optionally substituted isoxazolyl, optionally substituted thiazolyl, optionally substituted isothiazolyl, F, OH, hydroxyalkyl, O-alkyl, O-acyl, S-alkyl, NH₂, NH-alkyl, N(alkyl)₂, 1-pyrrolidinyl, 1-piperidinyl, 4-morpholinyl, NH-acyl, N(alkyl)-acyl, N₃, CO₂H, CO₂-alkyl, CONH₂, CONH-alkyl, CON(alkyl)₂, CN and CF₃;
Ar is selected from optionally substituted thienyl and optionally substituted phenyl;
a is 1 or 2, bis 1, 2 or 3; c is 1 or 2, d is 1, 2 or 3; e is 1, 2, 3 or 4; f is 1, 2 or 3 and g, h, i and are all independently 1 or 2;
provided that:
not more than one of A⁸, A⁹ and A¹⁰ is NH, N(CH₂)_{d}R⁷ or S;
A⁷ and A¹¹ are not both simultaneously N;
Neither A⁷ nor A¹¹ is N if one of A⁸, A⁹ and A¹⁰ is NH, N(CH₂)_{d}R⁷ or S;
if A¹⁰ is —CH═CH— then A⁸ is N, A⁹ is CH and both A⁷ and A¹¹ are C;
if A¹⁰ is not —CH═CH— then one of A⁸, A⁹ and A¹⁰ is NH, N(CH₂)_{d}R⁷ or S or one of A⁷ and A¹¹ is N;
not more than one of A¹⁴, A¹⁵ and A¹⁶ is NH, N—CH₃ or S;
A¹² and A¹³ are not both simultaneously N;
if one of A¹⁴, A¹⁵ and A¹⁶ is NH, N—CH₃ or S then A¹² and A¹³ are both C; and
one of A¹⁴, A¹⁵ and A¹⁶ is NH, N—CH₃ or S or one of A¹² and A¹³ is N.

2. A compound according to claim 1, or a pharmaceutically acceptable salt thereof, wherein at least one of R¹, R² and R³ is H and at least one is not H.

3. A compound according to claim 1, or a pharmaceutically acceptable salt thereof, wherein one of R¹, R² and R³ is selected from an alkyl group, F. C^{l} and Br and the others are H.

4. A compound according to claim 1, or a pharmaceutically acceptable salt thereof, wherein R¹ is selected from a methyl group and C^{l}, and R² and R³ are H.

5. A compound according to claim 1, or a pharmaceutically acceptable salt thereof, wherein X¹ is NH.

6. A compound according to claim 1, or a pharmaceutically acceptable salt thereof, wherein a is 1 and b is 2.

7. A compound according to claim 1, or a pharmaceutically acceptable salt thereof, wherein G¹ is a group according to general formula 3.

8. A compound according to claim 7, or a pharmaceutically acceptable salt thereof, wherein c is 2.

9. A compound according to claim 7, or a pharmaceutically acceptable salt thereof, wherein A¹ is CH₂ and A² is NH.

10. A compound according to claim 7, or a pharmaceutically acceptable salt thereof, wherein A¹ is NH or N-alkyl and A² is C(═O).

11. A compound according to claim 7, or a pharmaceutically acceptable salt thereof, wherein A³ is S and A⁴ and A⁵ are both CH.

12. A compound according to claim 7, or a pharmaceutically acceptable salt thereof, wherein A3 is —CH═CH— and A⁴ and A⁵ are both CH.

13. A compound according to claim 7, or a pharmaceutically acceptable salt thereof, wherein A³ is —CH═N— and A⁴ and A⁵ are both CH.

14. A compound according to claim 7, or a pharmaceutically acceptable salt thereof, wherein A³ is —CH═CH—, A⁴ is CH and A⁵ is N.

15. A compound according to claim 1, or a pharmaceutically acceptable salt thereof, wherein G¹ is a group according to general formula 6 or 7.

16. A compound according to claim 15, or a pharmaceutically acceptable salt thereof, wherein A³ is S and A⁴ and A⁵ are both CH.

17. A compound according to claim 15, or a pharmaceutically acceptable salt thereof, wherein A³ is —CH═CH— and A⁴ and A⁵ are both CH.

18. A compound according to claim 15, or a pharmaceutically acceptable salt thereof, wherein A³ is —CH═N— and A⁴ and A⁵ are both CH.

19. A compound according to claim 15, or a pharmaceutically acceptable salt thereof, wherein A³ is —CH═CH—, A⁴ is CH and A⁵ is N.

20. A compound according to claim 1, or a pharmaceutically acceptable salt thereof, wherein G¹ is a group according to general formula 4 or 6

21. A compound according to claim 20, or a pharmaceutically acceptable salt thereof, wherein A⁶ is NH.

22. A compound according to claim 20, or a pharmaceutically acceptable salt thereof, wherein A⁸ is NH or N—(CH₂)_{d}—R⁷.

23. A compound according to claim 22, or a pharmaceutically acceptable salt thereof, wherein A⁹ is N and A¹⁰ is CH.

24. A compound according to claim 1, or a pharmaceutically acceptable salt thereof, wherein R¹ is methyl or Cl, R² and R³ are both H and X¹ is NH.

25. A compound according to claim 1, or a pharmaceutically acceptable salt thereof, wherein R¹ is methyl or Cl, R² and R³ are both H, X¹ is NH, a is 1 and b is 2.

26. A compound according to claim 1, or a pharmaceutically acceptable salt thereof, wherein G¹ is a group according to general formula 6, A⁴, A⁵ and A¹⁰ are all CH, A⁶ is NH, A⁷ and A¹¹ are both C, A⁸ is N—(CH₂)_{d}—R⁷ and A⁹ is N.

27. A compound according to claim 1, or a pharmaceutically acceptable salt thereof, wherein R¹ is methyl or C¹, R² and R³ are both H, X¹ is NH, a is 1, b is 2, G¹ is a group according to general formula 6, A⁴, A⁵ and A¹⁰ are all CH, A⁶ is NH, A⁷ and A¹¹ are both C, AB is N—(CH₂)_{d}—R⁷ and A⁹ is N.

28. A compound according to claim 1 selected from
5-(4-(4-cyclopropylmethylpiperazine-1-carbonylaminomethyl)-3-methylbenzoyl)-1-methyl-4,10-dihydropyrazolo[5,4-b][1,5]benzodiazepine,
5-(4-(4-benzylpiperazine-1-carbonylaminomethyl)-3-methylbenzoyl)-1-methyl-4,10-dihydropyrazolo[5,4-b][1,5]benzodiazepine,
5-(4-(4-(3-hydroxybenzyl)piperazine-1-carbonylaminomethyl)-3-methylbenzoyl) 1-methyl-4,10-dihydropyrazolo[5,4-b][1,5]benzodiazepine,
5-(4-(4-(3-hydroxymethylbenzyl)piperazine-1-carbonylaminomethyl)-3-methylbenzoyl)-1-methyl-4,10-dihydropyrazolo[5,4-b][1,5]benzodiazepine,
1-methyl-5-(3-methyl-4-(4-(4-picolyl)piperazine-1-carbonylaminomethyl)benzoyl)-4,10-dihydropyrazolo[5,4-b][1,5]benzodiazepine,
5-(4-(4-(2-hydroxyethyl)piperazine-1-carbonylaminomethyl)-3-methylbenzoyl)-1-methyl-4,10-dihydropyrazolo[5,4-b][1,5]benzodiazepine,
1-methyl-5-(3-methyl-4-(4-(3-(methylthio)propyl)piperazine-1-carbonylaminomethyl)benzoyl)-4,10-dihydropyrazolo[5,4-b][1,5]benzodiazepine,
5-(4-(4-(2-aminoethyl)piperazine-1-carbonylaminomethyl)-3-methylbenzoyl)-1-methyl-4,10-dihydropyrazolo[5,4-b][1,5]benzodiazepine,
5-(4-(4-(2-hydroxyethyl)piperazine-1-carbonylaminomethyl)-3-methylbenzoyl)-1-methyl-4,10-dihydropyrazolo[4,5-c]pyrido[2,3-b][1,4]diazepine,
and pharmaceutically acceptable salts thereof.

29. At least one optical isomer of a compound or salt according to claim 1.

30. A pharmaceutical composition which comprises a compound, salt or isomer according to claim 1 as an active agent.

31. A pharmaceutical composition according to claim 30 which is a tablet or capsule for oral administration.

32. A pharmaceutical composition according to claim 30 which is for the treatment of male erectile dysfunction.

33. A use for a compound, salt or isomer according to claim 1, which is as a component in the manufacture of a pharmaceutical composition.

34. A use according to claim 33 wherein the pharmaceutical composition is to be used in the treatment of male erectile dysfunction.

35. A method of treating male or female sexual disorders which comprises the administration to a person in need of such treatment of an effective amount of a compound, salt or isomer according to claim 1.
